(19) United States
(12) Patent Application Publication
(10) Pub. No.: US 2026/0193672 A1
(43) Pub. Date: Jul. 9, 2026

(54) INDUCIBLE CELL SUICIDE SWITCHES AND THEIR USE IN CELL AND GENE THERAPIES

(71) Applicant: The General Hospital Corporation, Boston, MA (US)

(72) Inventors: Max Jan, Cambridge, MA (US); Robert T. Manguso, Jamaica Plain, MA (US); Marcela V. Maus, Lexington, MA (US); Ditsa Sarkar, Boston, MA (US)

(21) Appl. No.: 19/131,785

(22) PCT Filed: Nov. 21, 2023
(86) PCT No.: PCT/US2023/080756

§ 371 Date: May 21, 2025

Related U.S. Application Data
(60) Provisional application No. 63/384,507, filed on Nov. 21, 2022.

(51) Int. Cl. C12N 15/86; A61K 31/454; A61K 31/4545; A61K 40/11; A61K 40/31; C07K 14/47; C07K 16/00; C12N 5/0783; C12N 9/22

(52) U.S. Cl. CPC C12N 15/86; A61K 31/454; A61K 31/4545; A61K 40/11; A61K 40/31; C07K 14/47; C07K 16/00; C12N 5/0636; C12N 9/22; C12N 2510/00; C12N 2740/15043; C12N 2840/203

(57) ABSTRACT

Provided herein are small-molecule inducible cell suicide switches and their use in cell and gene therapies.

### CLAIM OF PRIORITY

This application claims the benefit of U.S. Provisional Application Ser. No. 63/384,507, filed on Nov. 21, 2022. The entire contents of the foregoing are incorporated herein by reference.

### TECHNICAL FIELD

Provided herein are small-molecule inducible cell suicide switches and their use in cell and gene therapies.

### BACKGROUND

Cell-based therapies are emerging as effective agents against cancer and other diseases. However, as autonomous “living drugs,” these therapies have the potential for toxic effects due to overactivity or adverse reactions.

### SUMMARY

Provided herein are expression constructs comprising a promoter driving expression of an anti-apoptotic transgene encoding a degron-tagged human inhibitor of caspase-activated DNase (ICAD) and a pro-apoptotic transgene encoding a human caspase-activated DNase (CAD), wherein the degron-tagged ICAD comprises a degron sequence at the C terminus of ICAD.

In some embodiments, the expression construct is multicistronic and comprises one promoter and one or more 2A or internal ribosome entry site (IRES) sequences between the anti-apoptotic transgene encoding degron-tagged ICAD and the pro-apoptotic transgene encoding CAD. In some embodiments, the promoter is a human elongation factor-1 alpha (EF1α) promoter. In some embodiments, the CAD comprises one or more mutations at residues corresponding to R11, K18, Y49, or E68 of SEQ ID NO:. In some embodiments, the CAD comprises one or more mutations selected from R11A, R11W, K18A, Y49W, and E68A, and combinations thereof. In some embodiments, the CAD comprises mutations R11W/K18A or R11W/E68A.

In some embodiments, the degron tag is a IKZF3 degron or dTAG. In some embodiments, the IKZF3 degron comprises a sequence at least 80%, 85%, 90%, 95%, or 90% identical to one of ZFP91-IKZF3 degron (FNVLMVHKRSHTGERPLQCEICGFTCRQKGNLLRHIKLHTGEKPFKCHLCNY ACQRRDAL (SEQ ID NO:3)); degron 913iK0 (FNVLMVHRRSHTGERPLQCEICGFTCRQRGNLLRHIRLHTGERPFRCHLCNY ACQRRDAL (SEQ ID NO:4)); or IKZF3 degron polypeptide 130-189 (FNVLMVHKRSHTGERPFQCNQCGASFTQKGNLLRHIKLHTGEKPFKCHLCN YACQRRDAL (SEQ ID NO:5)).

In some embodiments, the CAD comprises a sequence at least 80 identical to SEQ ID NO:1.

In some embodiments, the ICAD comprises a sequence at least 80%, 85%, 90%, 95%, or 90% identical to SEQ ID NO:2.

In some embodiments, the expression construct is in a viral vector, e.g., a lentivirus, adenovirus, or adeno-associated virus.

Also provided herein are cells expressing a transgene encoding a degron-tagged human inhibitor of caspase-activated DNase (ICAD), wherein the degron-tagged ICAD comprises a degron sequence at the C terminus of ICAD, and a pro-apoptotic transgene encoding human caspase-activated DNase (CAD), preferably wherein the degron-tagged ICAD and the CAD are expressed in an approximately 1:1 ratio. As used herein, “approximately” or “about” mean plus or minus 10%. Additionally, provided herein are cells comprising an expression construct as described herein, and optionally expressing the degron-tagged human ICAD and human CAD. In some embodiments, the cell further expresses a therapeutic transgene.

In some embodiments, the therapeutic transgene is a chimeric antigen receptor (CAR). In some embodiments, the cell is a T cell or a natural killer (NK) cell.

In some embodiments, the cell is a human cell.

Further, provided herein are methods of providing a cell therapy to a human subject, the method comprising administering to the subject a cell as described herein, e.g., a cell expressing a transgene encoding a degron-tagged human inhibitor of caspase-activated DNase (ICAD), wherein the degron-tagged ICAD comprises a degron sequence at the C terminus of ICAD, and a pro-apoptotic transgene encoding human caspase-activated DNase (CAD), preferably wherein the degron-tagged ICAD and the CAD are expressed in an approximately 1:1 ratio. Additionally, provided herein are cells comprising an expression construct as described herein, and optionally expressing the degron-tagged human ICAD and human CAD. In some embodiments, the cell further expresses a therapeutic transgene.

In some embodiments, the methods further comprise administering to the subject an effective amount of a small molecule controller of the degron, wherein the small molecule controller triggers degradation of the degron-tagged anti-apoptotic ICAD protein. In some embodiments, a) the degron is an IKZF3 degron and the small molecule controller comprises thalidomide or a thalidomide analog; or b) the degron is dTAG and the small molecule controller comprises dTag-13 (1-[(2S)-1-Oxo-2-(3,4,5-trimethoxyphenyl)butyl]-(2S)-2-piperidinecarboxylate (1R)-3-(3,4-dimethoxyphenyl)-1-[2-[2-[[6-[[2-(2,6-dioxo-3-piperidinyl)-2,3-dihydro-1,3-dioxo-1H-isoindol-4-yl]oxy]hexyl]amino]-2-oxoethoxy]phenyl]propyl ester.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-B: Other pairs of pro/anti-apoptotic proteins did not work as an effective suicide switch. (A) BCL-xL and BIM transgene pairs were toxic to Jurkat cells transduced to express these lentiviruses, as assessed by total cell number 48 hours after high-titer transduction. (B) Lenalidomide did not alter the growth and survival of cells transduced to express pairs of BCL-xL and variants of PUMA fused to an E3 recruitment domain 913iK0.

FIG. 2: C-terminal position of degron is required for efficient lentiviral transduction. Transduction efficiency of Jurkat cells with the indicated lentiviral supernatant is shown.

FIG. 3: Variations in CAD modify performance of CAD/ICAD suicide switch. Primary human T cell four day growth assay comparing vector control, and then three modifications of ICAD-degron 2A CAD expression, differentiated by the CAD sequences. The p.R11W mutation enhances drug-independent cell fitness. The ICAD-degron 2A CAD p.R11W is referred to as the lenalidomide “kill switch” or “suicide switch” in prior and subsequent slides.

FIG. 4: Promoter strength modifies ICADdegron-CAD suicide switch efficiency. Enhanced DNA damage response was seen from the construct using the EF1alpha promoter.

FIGS. 5A-H: An ICAD-degron/CAD transgene pair acted as a lenalidomide-inducible suicide switch.
A. Schema
B. Assessment of GFP-ICAD-degron reporter fluorescence in Jurkat cells. Left—exposure to a range of drug concentrations overnight. Right—timecourse after 1000 nM lenalidomide addition.
C. Schema for lenalidomide suicide switch. Gamma H2AX fluorescence as a marker of DNA damage assessed across a range of drug concentrations administered overnight (left) or assessed as a timecourse after 1000 nM lenalidomide addition.
D. Western blot to assess the abundance of endogenous and transgenic overexpressed proteins with and without 4 hours of exposure to 1000 nM lenalidomide addition.
E. (Left) Competitive growth assay of untransduced versus vector control or CAD/ICAD suicide switch primary human T cells. (Right) mTagBFP2 MFI of mTagBFP2+ cells, demonstrating the selective survival of mTagBFP® cells. (BFP is blue fluorescent protein)
F. Cell depletion (normalized to untreated cells) of primary human CAD/ICAD versus iCasp9 suicide switch T cells, across dose ranges of AP1903 and lenalidomide.
G. Representative flow plot of CAD/ICAD suicide switch cells with/without lenalidomide.
H. Representative flow plot of iCasp9 suicide switch cells with/without lenalidomide.

FIGS. 6A-F: Control of CAR T cell function with lenalidomide suicide switch.
A. Schema of dual-vector CAR and ICAD-degron/CAD suicide switch strategy.
B. Depletion of ICAD-degron/CAD suicide switch-positive primary human T cells after 1 uM lenalidomide treatment.
C. Depletion of sorted CAR+ ICAD-degron/CAD suicide switch+primary human T cells across dose range of lenalidomide.
D. Incucyte live cell imaging co-culture assay with NALM6 tumor cells and sorted dual-vector primary human CAR T cells. Drug-dependent inhibition of anti-tumor effect.
E. Drug-dependent inhibition or ablation of CAR T cell expansion in same Incucyte assay.
F. Incucyte live cell imaging co-culture assay with NALM6 tumor cells and single lentivector ICAD-degron/CAD/CAR primary human T cells. Drug-dependent inhibition of anti-tumor effect.

FIG. 7: ICAD-degron CAD suicide switch design was generalizable to multiple degron systems. Three different degron tags (SMASH, dTAG (degradation TAG), lenalidomide ZFP91-IKZF3 “superdegron”) were fused to ICAD to facilitate ICAD degradation.

FIG. 8: Further substitutions in CAD reduced the fitness cost of suicide switch overexpression without diminishing lenalidomide-induced depletion. Competitive growth assay of modified lenalidomide suicide switches in Jurkat cells, with 1 uM lenalidomide (dashed lines) or without (solid lines), normalized to % transduced on day 0.

### DETAILED DESCRIPTION

Definitive safety systems are needed for next-generation cellular immunotherapies. Already, clinically approved CAR T cell therapies can cause severe treatment-refractory T cell hyperactivation syndromes (1). Unexpected disease- or product-specific toxicities remain difficult to manage, especially in early stage clinical trials (2). As effective cellular immunotherapies emerge for solid tumors (3), anatomic challenges, imperfect target antigens, and the need for more potent tumor microenvironment-resistant immune effector cells all increase the need for enhanced safety controls. Moreover, an enhanced therapeutic window will also be needed as cellular immunotherapies emerge as effective agents for indications beyond cancer (4).

Molecularly diverse suicide switches have been developed and deployed clinically (5), yet such safety controls have not become standard elements of cell therapy design, in part due to limited performance characteristics, the use of non-approved small molecule controllers, and the use of non-human sequences. An ideal suicide switch system would not add onerous cell manufacturing complexity, not reduce engineered cell fitness, eliminate engineered cells upon induction by an approved drug controller, be a compact, all-human genetic element, and be amenable to a range of promoter strengths and cell states.

Here we present the development of a suicide switch controlled by small molecules, such as lenalidomide and its analogs. Lenalidomide is a frontline therapy for people with hematologic malignancies including multiple myeloma and myelodysplastic syndrome with deletion of chromosome 5q that in broad clinical testing has been found to be safe but with limited efficacy targeting solid tumors (6). Lenalidomide acts as a molecular glue to recruit disease-relevant neosubstrate proteins to the CRL4^{CRBN} E3 ubiquitin ligase, resulting in targeted protein degradation (7, 8). Engineered zinc finger domains from neosubstrate proteins can be used as “degron” tags for lenalidomide-inducible degradation of CARs and other fusion proteins (9-12). We envisioned a suicide switch composed of a single promoter, multi-cistronic transgene pair of a pro-apoptotic protein and a degron-tagged anti-apoptotic protein (13). Caspase-activated DNase (CAD) and its inhibitor (ICAD) are a uniquely promising pair for this molecular architecture (14, 15). ICAD serves as a chaperone for CAD folding and then locks CAD in an inactive heterodimer (16-18). Upon apoptosis induction, Caspase 3 cleaves ICAD, allowing unopposed CAD to homodimerize and cleave the genome, leading to cell death (19). We hypothesized that a suicide switch system composed of CAD and a degron-tagged ICAD could be expressed with little-to-no impact on cell fitness, until lenalidomide-induced degradation of ICAD liberates CAD to induce cell death. Shown herein is the development of an exemplary lenalidomide-inducible suicide switch and deployment as a safety system for CAR T cell therapy.

As one example, described herein is a ICAD-degron/CAD lenalidomide-inducible suicide switch that enabled rapid >90% cell depletion with subtherapeutic lenalidomide concentrations. We investigated potential genetic resistance mechanisms and found only the expected dependency on the E3 ligase machinery required for lenalidomide-mediated neosubstrate protein degradation. As a first translational proof of concept, the ICAD-degron/CAD switch was well-tolerated by CAR T cells and enabled robust lenalidomide-dependent cell depletion and cessation of effector functions.

Existing suicide switch designs have strengths and weaknesses. The iCasp9 system is clinically validated as an approach to mitigate graft-versus-host disease in the setting of haploidentical hematopoietic cell transplantation by depleting graft T cells (24). iCasp9 relies on AP1903, a chemical dimerizer not yet approved for clinical use, as well as a relatively narrow acceptable expression range with acceptably low drug-independent switch leakiness and also sufficient drug-dependent cell elimination. The Herpes Simplex Virus thymidine kinase transgene induces cell death upon addition of the prodrug ganciclovir and conversion into an active DNA replication inhibitor, and is also clinically validated as a strategy to control GvHD after donor lymphocyte infusion (25), although the use of a viral protein may lead to immunologic rejection. The expression of surface epitopes for monoclonal antibody therapeutics such as rituximab (26) or cetuximab (27) rely on cell-extrinsic effector mechanisms that may be disrupted in people with advanced cancers, and also acts by exposing patients to the long-term pharmacokinetics and side effect profile of the paired antibody therapeutic. The more recently described metabolic UMPS-knockout auxotrophy suicide switch is uniquely transgene-free (28), although it does require the additional complexity of genome editing. Each of these systems is likely to best align with different translational use cases. The principal limitation of ICAD-degron/CAD is its 2.2 kilobase transgene size, although this genetic payload size was still compatible with high titer transduction of primary human cells with one lentivector encoding for both ICAD-degron/CAD and a CAR. Indeed, co-expression of ICAD-degron/CAD and a therapeutic protein (here demonstrated with CAR19) from a single lentivector, single promoter-driven tricistronic transgene could be an added safety feature. Transgene silencing resulting in reduced ICAD-degron/CAD abundance would also presumably silence the toxicity-associated therapeutic element. ICAD-degron/CAD provided the advantages of robust >90% cell depletion induced by an approved and well-tolerated drug, composition of all-human sequences, and feasibility with standard lentiviral transgene delivery using a strong constitutive promoter. These features together can be used in diverse cell engineering and clinical use cases.

ICAD-degron/CAD switches have broad applicability beyond CAR T cell therapy. While CAD and ICAD expression may be optimized for additional cell types, both the CAD and ICAD stoichiometric inhibitor mechanism and the requisite CRL4^{CRBN} E3 ligase components are almost ubiquitously expressed across cell types (29). ICAD-degron/CAD has immediate translational potential to serve as a safety switch in diverse gene- and cell-based therapies (30, 31).

Thus, provided herein are constructs comprising cell suicide switches, and cells that express the constructs, and optionally also express one or more therapeutic proteins, and their use in gene- and cell-based therapy.

### Inducible Suicide Switches

The cell suicide switches (also referred to as kill switches) described herein comprise 1) a degron-tagged anti-apoptotic transgene and 2) a pro-apoptotic transgene; a small molecule controller, e.g., a thalidomide analog, is used to turn the switch “on” resulting in depletion of the degron-tagged anti-apoptotic protein. The degron-tagged anti-apoptotic protein inhibits the function of the pro-apoptotic factor, in some instances as a stoichiometric binding partner and inhibitor. For example, the switches can employ an IKZF3 degron protein sequence previously described (see WO2019089592). Fusion proteins including this degron tag are recruited to the CRL4^{CRBN} E3 ubiquitin ligase upon addition of molecular glue thalidomide analog drugs, resulting in fusion protein ubiquitination and proteasomal degradation. Thalidomide analogs including lenalidomide and pomalidomide are clinically approved anti-cancer drugs. Upon thalidomide analog addition, the depletion of the degron-tagged anti-apoptotic protein results in an excess of pro-apoptotic factors, irreversibly triggering cell death.

### Degron Tags

Cereblon (CRBN) is a 442 amino acid protein that forms an E3 ubiquitin ligase complex with damaged DNA binding protein 1 (DDB1), Cullin-4A (CUL4A) and regulator of cullins 1 (ROC1; Angers et al. Nature 443: 590-593). This complex ubiquitinates a number of other proteins. Preclinical studies identified CRBN as a direct molecular target for the teratogenicity of thalidomide. CRBN binds directly to thalidomide analog affinity beads and is linked to the teratogenic effects of thalidomide in zebrafish and chicks (Ito et al. Science 327: 1345-1350). It was also shown that thalidomide, lenalidomide, and pomalidomide each binds to CRBN in vitro (Lopez—Girona et al. Leukemia 26: 2326-2335). An exemplary sequence of Homo sapiens CRBN polypeptide can be found at GenBank Accession No. AAH17419.1, and minimal forms are described in WO2019089592. Known CRBN substrates include: Ikaros (IKZF1), Aiolos (IKZF3) casein kinase 1α (Ck1α), Homeobox protein Meis2 (MEIS2), E3 ubiquitin-protein ligase (ZFP91), Eukaryotic peptide chain release factor GTP-binding subunit ERF3A (GSPT1), and Glutathione synthetase (GSS); see WO2019089592.

In some embodiments, the degron comprises a “superdegron” also known as ZFP91-IKZF3 degron, comprising the sequence FNVLMVHKRSHTGERPLQCEICGFTCRQKGNLLRHIKLHTGEKPFKCHLCNY ACQRRDAL (SEQ ID NO:3); degron 913iK0, comprising the sequence FNVLMVHRRSHTGERPLQCEICGFTCRQRGNLLRHIRLHTGERPFRCHLCNYA CQRRDAL (SEQ ID NO:4); or IKZF3 degron polypeptide, amino acids 130-189 FNVLMVHKRSHTGERPFQCNQCGASFTQKGNLLRHIKLHTGEKPFKCHLCNY ACQRRDAL (SEQ ID NO:5). See WO2019089592 for other IKZF3 degron sequences. In some embodiments, the degron sequence is at least 80%, 85%, 90%, 95%, 97%, 98%, or 99% identical to a sequence set forth above, or has up to 1, 2, 3, 4, 5, 6, 7, 8, 9, or 10 amino acid substitutions or deletions as compared to a sequence set forth herein. In some embodiments, the substitutions are conservative substitutions.

Alternatively, the degradation TAG (dTAG) system, which pairs a degrader of FKBP12^{F36V} with expression of FKBP12^{F36V} in-frame with the anti-apoptotic transgene, can be used. See, e.g., Nabet et al., Nat Chem Biol. 2018 May; 14(5): 431-441. dTag also works by activating CRBN.

### Anti-Apoptotic Transgenes and Pro-Apoptotic Transgenes

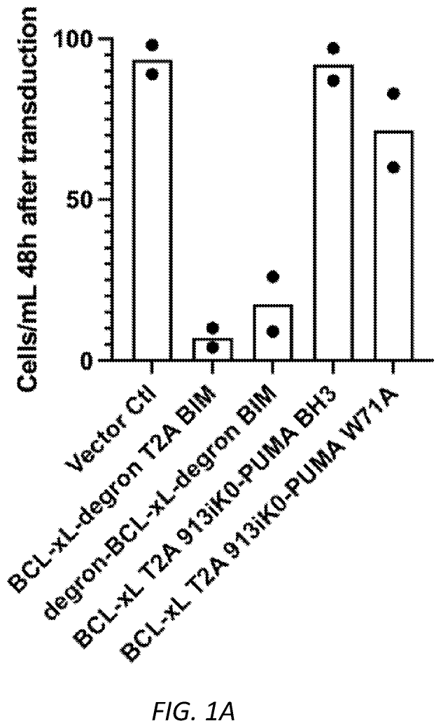

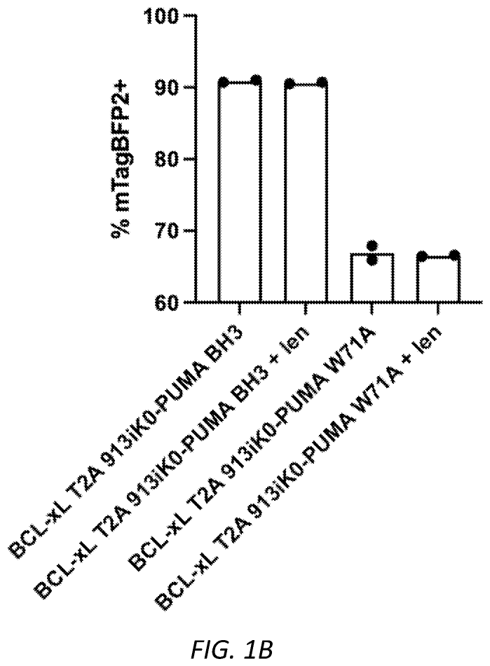

In the present constructs, a pair of transgenes are expressed: 1) a pro-apoptotic gene, and 2) a controller-responsive degron-tagged anti-apoptotic gene. These would constitute a stoichiometric pair that would be inert until the controller (e.g., lenalidomide) is added, resulting in anti-apoptotic protein degradation, an excess of pro-apoptotic proteins, activated apoptotic signaling, and cell death. Not all embodiments of this idea worked (see Example 1, FIGS. 1A-B). The pair of BIM and BCL-xL tagged with N-terminal or C-terminal degron tags was immediately toxic irrespective of lenalidomide addition. A related idea, an anti-apoptotic protein (BCL-xL and a zinc finger-tagged BH3 domain that could in theory recruit pro-apoptotic binding parter proteins to CRL4CRBN upon lenalidomide addition did not induce cell death upon lenalidomide addition (right). Thus, the present constructs use paired pro- and anti-apoptotic transgenes caspase-activated deoxyribonuclease (CAD) and its inhibitor (ICAD), preferably a degron-tagged ICAD that comprises a degron sequence at the C terminus of ICAD. CAD degrades chromosomal DNA during apoptosis, and is held as an inactive monomer by association with ICAD. In the present constructs, C-terminally degron-tagged ICAD is used.

An exemplary sequence of human CAD is available at NCBI Reference Sequence No: NP_001269598.1, and the following (bold letters indicate residues for possible mutation):

(SEQ ID NO: 1)
MLQKPKSVKLRALRSPRKFGVAGRSCQEVLRKGCLRFQLPERGSRLCLY
EDGTELTEDYFPSVPDNAELVLLTLGQAWQGYVSDIRRFLSAFHEPQVG
LIQAAQQLLCDEQAPQRQRLLADLLHNVSQNIAAETRAEDPPWFEGLES
RFQSKSGYLRYSCESRIRSYLREVSSYPSTVGAEAQEEFLRVLGSMCQR
LRSMQYNGSYFDRGAKGGSRLCTPEGWFSCQGPFDMDSCLSRHSINPYS
NRESRILFSTWNLDHIIEKKRTIIPTLVEAIKEQDGREVDWEYFYGLLF
TSENLKLVHIVCHKKTTHKLNCDPSRIYKPQTRLKRKQPVRKRQ

An exemplary sequence of human ICAD is available at GenBank Accession No. NP_004392.1, and the following:

(SEQ ID NO: 2)
MEVTGDAGVPESGEIRTLKPCLLRRNYSREQHGVAASCLEDLRSKACDI
LAIDKSLTPVTLVLAEDGTIVDDDDYFLCLPSNTKFVALASNEKWAYNN
SDGGTAWISQESFDVDETDSGAGLKWKNVARQLKEDLSSIILLSEEDLQ
MLVDAPCSDLAQELRQSCATVQRLQHTLQQVLDQREEVRQSKQLLQLYL
QALEKEGSLLSKQEESKAAFGEEVDAVDTGISRETSSDVALASHILTAL
REKQAPELSLSSQDLELVTKEDPKALAVALNWDIKKTETVQEACERELA
LRLQQTQSLHSLRSISASKASPPGDLQNPKRARQDPT

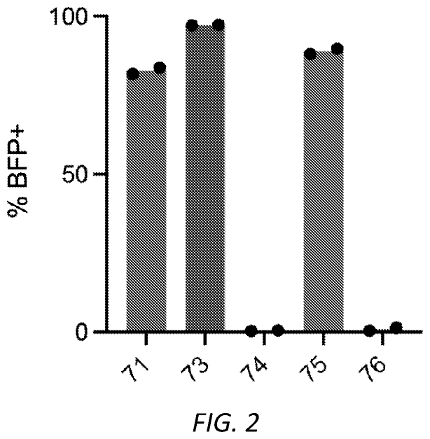

As shown herein, the degron is preferably placed on the C terminus of ICAD (ICAD-degron); this may be because the degron-ICAD conformation is unstable, resulting in toxicity both to the viral producer cells and any transduced cells (FIG. 2).

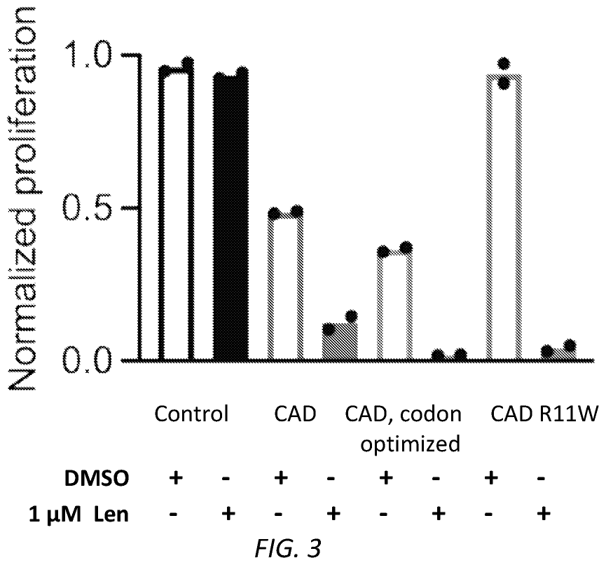

When we created an ICAD-degron-T2A-CAD multi-cistronic putative suicide switch, the amino acid and nucleotide sequence of CAD was critical to drug-independent fitness cost and drug-induced depletion (FIG. 3). A p.R11W mutation that was inadvertently created during molecular cloning, identified, and retained in the analysis, turned out to be critical to optimizing switch stability and drug-dependent cell depletion.

The CAD and ICAD used in the present methods can include one or more mutations that enhance ICAD-degron:CAD switch performance, e.g., that enhance drug-independent cell fitness, including mutations in CAD at residues R11, K18, Y49, and E68, such as R11A, R11W, K18A, Y49W, and E68A, as well as combinations thereof such as R11W/K18A, and R11W/E68A. In some embodiments, the CAD includes a R11W mutation in CAD.

A preferred construct includes ICAD-degron—2A—CAD p.R11W.

### Small Molecule Controllers

The present methods include administration of an effective amount of a small molecule controller of the degron, which triggers degradation of the degron-tagged anti-apoptotic ICAD protein and allows the pro-apoptotic CAD to degrade genomic DNA in the cell.

For IKZF3 degrons, thalidomide and thalidomide analogs can be used, including 5-hydroxythalidomide, thalidomide, lenalidomide, pomalidomide, avadomide (CC-122), or CC-885; see, e.g., Sperling et al., Blood. 2019 Jul. 11; 134(2): 160-170, and WO2019089592.

For the dTag system, a dTag compound is used, e.g., dTag-13 (1-[(2S)-1-Oxo-2-(3,4,5-trimethoxyphenyl)butyl]-(2S)-2-piperidinecarboxylate (1R)-3-(3,4-dimethoxyphenyl)-1-[2-[2-[[6-[[2-(2,6-dioxo-3-piperidinyl)-2,3-dihydro-1,3-dioxo-1H-isoindol-4-yl]oxy]hexyl]amino]-2-oxoethoxy]phenyl]propyl ester, Tocris); others, including dTAG-7, dTAG-48, and dTAG-51, can also be used (see Nabet et al., Nat Chem Biol. 2018 May; 14(5): 431-441).

### Expression Constructs

Sequences encoding the cell suicide switches can be delivered to a cell using an expression construct. These expression constructs can be administered in any effective carrier, e.g., any formulation or composition capable of effectively delivering the component gene to the cells. A nucleic acid encoding the selected protein can be inserted in an expression vector, to make an expression construct. A number of suitable vectors are known in the art, e.g., viral vectors including recombinant retroviruses, lentiviruses, adenovirus, adeno-associated virus, and herpes simplex virus-1, adenovirus-derived vectors, or recombinant bacterial or eukaryotic plasmids. In some embodiments, the vector is a lentiviral vector; see, e.g., Poletti and Mavilio, Viruses. 2021 August; 13(8): 1526; Labbe et al., Viruses. 2021 August; 13(8): 1528.

Exemplary expression constructs can include: a coding region; a promoter sequence, e.g., a promoter sequence that restricts expression to a selected cell type, a conditional promoter, or a strong general promoter; an enhancer sequence; untranslated regulatory sequences, e.g., a 5′untranslated region (UTR), a 3′UTR; a polyadenylation site; and/or an insulator sequence. Such sequences are known in the art, and the skilled artisan would be able to select suitable sequences. See, e.g., Current Protocols in Molecular Biology, Ausubel, F. M. et al. (eds.) Greene Publishing Associates, (1989), Sections 9.10-9.14; Vancura (ed.), Transcriptional Regulation: Methods and Protocols (Methods in Molecular Biology (Book 809)) Humana Press; 2012 edition (2011) and other standard laboratory manuals. Protocols for producing recombinant viruses and for infecting cells in vitro or in vivo with such viruses are known in the art; examples can be found in Ausubel, et al., eds., Gene Therapy Protocols Volume 1: Production and In Vivo Applications of Gene Transfer Vectors, Humana Press, (2008), pp. 1-32; Ghosh et al., Viral Vector Systems for Gene Therapy: A Comprehensive Literature Review of Progress and Biosafety Challenges, Applied Biosafety. March 2020. 7-18; and other laboratory manuals.

The vectors can include, e.g., inverted terminal repeats (ITRs) (for AAV); promoters, enhancers (e.g., CMV enhancer), other cis-regulatory elements, and/or capsid serotype variants that control and drive expression of the MCOLN1 protein. With regard to promoters, vectors can include promoters that drive expression in many cell types (e.g., human β-actin, human elongation factor-1 alpha (EF1α), chicken β-actin combined with cytomegalovirus early enhancer, cytomegalovirus (CMV), simian virus 40 (SC40), herpes simplex virus thymidine kinase (HSVTK), PGK, CAG, sCAG, or CASI promoters). Other cis-regulatory elements can include posttranscriptional regulatory elements; 2A elements; IRES sequences; polyadenylation sequences; and/or an intron.

Posttranscriptional regulatory elements can include HBV Posttranscriptional Regulatory Element (HPRE), woodchuck hepatitis virus post-transcriptional regulatory element (WPRE) or variants thereof (e.g., WPRE2, WPRE3 see, e.g., Kalev-Zylinska M L, During MJJ Neurosci. 2007 Sep. 26; 27(39):10456-67; Zanta-Boussif et al., Gene Therapy (16): 605-619 (2009); Choi et al., Mol Brain. 7:17 (2014); U.S. Pat. No. 6,136,597).

Preferably, multicistronic vectors can be used, in which the vector includes sequences encoding at least the CAD and ICAD-degron, and optionally a therapeutic transgene as well. To create multicistronic constructs, one or more 2A or IRES sequences can be used. 2A sequences are typically 18-22 aa-long peptides that share a core sequence motif of DxExNPGP (SEQ ID NO:6) and induce ribosomal skipping during translation of a protein in a cell. Commonly used 2A sequences include P2A, E2A, F2A and T2A. F2A is derived from foot-and-mouth disease virus 18; E2A is derived from equine rhinitis A virus; P2A is derived from porcine teschovirus-1 2A; T2A is derived from thosea asigna virus 2A. See, e.g., Lewis et al., J Neurosci Methods. 2015 Dec. 30; 256: 22-29; Liu et al., Sci Rep. 2017 May 19; 7(1):2193. Internal ribosome entry site (IRES) sequences can also be used. Typical IRES sequences include the IRES sequence of encephalomyocarditis virus or vascular endothelial growth factor and type 1 collagen-inducible protein (VCIP). The 2A or IRES sequences can be placed in between coding sequences (e.g., between the ICAD-degron and CAD coding sequences, and any other coding sequences, e.g., reporter genes or therapeutic transgenes).

Exemplary polyadenylation sequences, which include SV40, human growth hormone (hGH), bovine growth hormone (bGH), synthetic polyadenylation (spA), and rbGlob, preferably include the sequence motif AAUAAA that promotes both polyadenylation and termination (Buck and Wijnholds, Int J Mol Sci. 2020 June; 21(12): 4197).

Introns can include SV40 intron, FIX truncated intron 1; β-globin SD/immunoglobin heavy chain SA; Adenovirus SD/immunoglobulin SA; SV40 late SD/SA (19S/16S); Hybrid adenovirus SD/IgG SA; or minute virus of mice (MVM) intron (see Powell and Rivera-Soto, Discov Med. 2015 January; 19(102): 49-57.

Also provided herein are compositions and formulations comprising the vectors, e.g., in a sterile carrier. Formulation of pharmaceutically-acceptable excipients and carrier solutions is well-known to those of skill in the art. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells.

Also provided herein are methods of making the expression constructs, as well as cells comprising the expression constructs. Methods for obtaining recombinant expression constructs having a desired sequence are well known in the art. Typically the methods involve culturing a host cell that contains a nucleic acid sequence encoding an expression construct. For lentiviral vectors, see, e.g., Poletti and Mavilio, Viruses. 2021 August; 13(8): 1526; Labbe et al., Viruses. 2021 August; 13(8): 1528.

### Cells

Further provided herein are cells that express the cell suicide switches described herein; the cells can also be engineered in other ways, optionally to express a therapeutic transgene or to increase, decrease, knock out, or otherwise alter expression of an endogenous gene. For example, the cells can also express a CAR, or can be engineered to lack expression of functional TGF-beta receptor or express a non-functional TGF-beta receptor; see, e.g., Bollard, C. J., et al., (2002) Blood 99:3179-3187; Bollard, C. M., et al., (2004) J. Exptl. Med. 200:1623-1633. Cells for use in cell transplantation can also be used, e.g., in case of development of severe graft-versus-host disease (GVHD).

In the event of an adverse event or reaction, the cells can be triggered to apoptose by administration of the controller.

A number of cell types can be used in the present methods and compositions, and the choice of cell type can be made depending on their ultimate purpose. Primary and secondary cells to be genetically engineered to express the constructs described herein can be obtained from a variety of tissues and can include cell types that can be maintained and propagated in culture. For example, primary and secondary cells used in cell therapy can include T cells, B cells, natural killer (NK) cells, adipose cells or adipose progenitor cells, human umbilical vein cells (HUVECs), pancreatic islet R cells, keratinocytes, epithelial cells (e.g., mammary epithelial cells, intestinal epithelial cells), endothelial cells, glial cells, neural cells, formed elements of the blood (e.g., lymphocytes, bone marrow cells), muscle cells (myoblasts) and precursors of these somatic cell types, kidney cells, vascular cells, pericytes, chondrocytes, mesenchymal stem cells, hematopoietic stem cells, dendritic cells, retinal pigment epithelial (RPE) cells, retinal stem cells, iPSC-derived cells, neural cells or neural progenitor cells, cord blood stem cells, fibroblasts, and stromal cells, see, e.g., Bashor et al., Nat Rev Drug Discov. 2022 September; 21(9):655-675.

Primary cells are preferably obtained from the individual to whom the genetically engineered primary or secondary cells will be administered (i.e., are autologous). However, primary cells may be obtained from a donor (i.e., an individual other than the recipient).

The term “primary cell” includes cells present in a suspension of cells isolated from a vertebrate tissue source (prior to their being plated, i.e., attached to a tissue culture substrate such as a dish or flask), cells present in an explant derived from tissue, both of the previous types of cells plated for the first time, and cell suspensions derived from these plated cells. The term “secondary cell” or “cell strain” refers to cells at all subsequent steps in culturing. Secondary cells are cell strains which consist of primary cells which have been passaged one or more times.

Primary or secondary cells of vertebrate, particularly mammalian, origin can be transfected with an exogenous nucleic acid sequence, which includes a nucleic acid sequence encoding a construct as described herein, and produce the encoded product stably and reproducibly in vitro and in vivo, over extended periods of time.

Vertebrate tissue can be obtained by standard methods such a punch biopsy or other surgical methods of obtaining a tissue source of the primary cell type of interest. For example, a biopsy can be used to obtain tissue as a source of primary cells. A mixture of primary cells can be obtained from the tissue, using known methods, such as enzymatic digestion or explanting. If enzymatic digestion is used, enzymes such as collagenase, hyaluronidase, dispase, pronase, trypsin, elastase and chymotrypsin can be used.

The resulting primary cell mixture can be transfected directly, or it can be cultured first, removed from the culture plate and resuspended before transfection is carried out. Primary cells or secondary cells are combined with exogenous nucleic acid sequence to, e.g., stably integrate into their genomes, and treated in order to accomplish transfection. As used herein, the term “transfection” includes a variety of techniques for introducing an exogenous nucleic acid into a cell including calcium phosphate or calcium chloride precipitation, microinjection, DEAE-dextrin-mediated or polyethylenimine transfection, lipofection sonoporation or electroporation, all of which are routine in the art.

Transfected primary or secondary cells undergo sufficient numbers of doubling to produce either a clonal cell strain or a heterogeneous cell strain of sufficient size to provide the therapeutic protein to an individual in effective amounts. The number of required cells in a transfected clonal heterogeneous cell strain is variable and depends on a variety of factors, including but not limited to, the use of the transfected cells, the functional level of the exogenous DNA in the transfected cells, the site of implantation of the transfected cells (for example, the number of cells that can be used is limited by the anatomical site of implantation), and the age, surface area, and clinical condition of the patient.

The transfected cells, e.g., cells produced as described herein, can be introduced into an individual to whom the product is to be delivered. Various routes of administration and various sites (e.g., renal sub capsular, subcutaneous, central nervous system (including intrathecal), intravascular, intrahepatic, intrasplanchnic, intraperitoneal (including intraomental), or intramuscular implantation) can be used. Once implanted in an individual, the transfected cells produce the product encoded by the heterologous DNA or are affected by the heterologous DNA itself.

### Therapeutic Transgenes

The cells described herein can also express one or more additional exogenous genes, e.g., therapeutic transgenes, e.g., for use in cell or gene therapy. As one example, the cells can be T cells in which T cell receptors (TCRs) are engineered to re-program the T cell with a new specificity, e.g., the specificity of a monoclonal antibody; this is generally referred to as a chimeric antigen receptor or CAR. The engineered TCR may make the T cells specific for malignant cells and therefore useful for cancer immunotherapy. For example, the T cells may recognize cancer cells expressing a tumor antigen, such as a tumor associated antigen that is not expressed by normal somatic cells from the subject tissue. Thus, the CAR-modified T or NK cells (also known as CAR-T and CAR-NK cells) can be used for adoptive T cell therapy of, for example, cancer patients (see Mohanty et al., Oncol. Rep. 2019; 42:2183-2195). See, e.g., U.S. Pat. No. 9,089,520.

Other therapeutic transgenes can also be used, e.g., cytokines, transcription factors, epigenetic regulators, co-regulatory ligands, receptors, and genome editing reagents such as TALEs, zinc fingers, CRISPR/Cas nucleases or nickases, base editors, prime editors, and so on; transforming growth factor-β (TGFβ), e.g., in chondrocytes; see, e.g., Medicines in Development for Cell and Gene Therapy 2020 Drug List, available at phrma.org/resource-center/Topics/Medicines-in-Development/Medicines-in-Development-for-Cell-and-Gene-Therapy-2020-Drug-List. Exemplary transgenes include BDNF, brain-derived neurotrophic factor; CFTR, cystic fibrosis transmembrane conductance regulator; CNGB3, cyclic nucleotide gated channel B3; GNAT, guanine nucleotide transducing; MMP-3, matrix metalloproteinase 3; ND4, NADH dehydrogenase protein subunit 4; RS1, retinoschisis 1; sFLOT01, fusion protein of VEGF and the Fc portion of the human IgG1; checkpoint inhibitory antibodies including anti-PD1, anti-PDL1, and anti-CTLA-4 antibody; FGF4, fibroblast growth factor 4; FGF21, fibroblast growth factor 21; FIX, factor IX; FVIII, factor VIII; hAAT, human alpha-1-antitrypsin; GAA, acid α-glucosidase; HbA, hemoglobin; HSV-TK, herpes simplex virus-thymidine kinase; KCNH2, I(Kr) potassium channel alpha subunit; HGF, human growth factor; VEGF, vascular endothelial growth factor; and insulin. See, e.g., Lundstrom, Viruses. 2023 March; 15(3): 698.

The therapeutic transgenes can be expressed from a separate construct, or from the same construct as the suicide switch (e.g., a multicistronic construct).

### Exemplary Sequences

In some embodiments, the sequence of a protein or nucleic acid used in a composition or method described herein is at least 80%, 85%, 90%, 95%, 97%, 98%, or 99% identical to a sequence set forth herein. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

In some embodiments, the sequence of a protein or nucleic acid used in a composition or method described herein has up to 1, 2, 3, 4, 5, 6, 7, 8, 9, or 10 amino acid substitutions or deletions as compared to a sequence set forth herein. In some embodiments, the substitutions are conservative substitutions.

BCL-XL
(SEQ ID NO: 7)
ATGTCTCAGTCAAATAGGGAACTCGTGGTCGATTTTTTGAGCTACAAACTT
AGTCAAAAAGGATATAGCTGGTCTCAGTTTTCCGATGTTGAGGAGAACAG
AACGGAAGCGCCCGAGGGGACTGAAAGCGAAATGGAAACTCCGTCAGCC
ATCAACGGTAATCCTTCCTGGCACCTCGCTGATTCACCTGCAGTCAATGGA
GCAACAGGACATTCATCATCACTCGACGCAAGAGAAGTTATTCCGATGGC
GGCTGTAAAGCAGGCATTGCGGGAAGCGGGTGACGAATTTGAGTTGAGGT
ACAGGAGGGCTTTCTCTGACCTGACTAGCCAACTGCATATTACGCCAGGC
ACTGCTTACCAGAGTTTTGAACAAGTGGTAAATGAACTCTTTAGAGATGG
GGTCAACTGGGGACGGATAGTTGCTTTTTTCAGCTTCGGCGGAGCCTTGTG
TGTAGAGAGCGTGGACAAGGAAATGCAGGTTCTCGTCAGCAGGATTGCAG
CCTGGATGGCGACTTACTTGAATGATCACCTCGAACCATGGATTCAAGAA
AATGGTGGTTGGGACACGTTCGTGGAACTCTACGGCAATAATGCCGCAGC
AGAAAGCCGCAAAGGCCAAGAAAGGTTCAACCGATGGTTTCTTACCGGA
ATGACAGTCGCTGGCGTTGTGCTCCTTGGGTCACTCTTTTCACGGAAG
(SEQ ID NO: 8)
MSQSNRELVVDFLSYKLSQKGYSWSQFSDVEENRTEAPEGTESEMETPSAIN
GNPSWHLADSPAVNGATGHSSSLDAREVIPMAAVKQALREAGDEFELRYRR
AFSDLTSQLHITPGTAYQSFEQVVNELFRDGVNWGRIVAFFSFGGALCVESVD
KEMQVLVSRIAAWMATYLNDHLEPWIQENGGWDTFVELYGNNAAAESRKG
QERFNRWFLTGMTVAGVVLLGSLFSRK
BIM
(SEQ ID NO: 9)
ATGGCAAAGCAGCCCTCAGATGTGTCTTCCGAATGCGACAGGGAAGGACG
CCAGCTTCAGCCTGCTGAGCGGCCCCCTCAACTCAGACCGGGAGCGCCAA
CTTCACTCCAAACAGAACCCCAAGGCAATCCCGAGGGTAACCACGGAGG
GGAAGGTGATTCATGTCCGCATGGTTCCCCACAGGGACCGTTGGCGCCAC
CGGCGTCaCCAGGTCCTTTCGCAACTAGGTCACCGCTCTTCATCTTCATGC
GGCGATCTTCCCTTTTGAGTCGCTCAAGCAGCGGTTACTTCAGCTTCGATA
CCGACCGATCACCGGCACCAATGAGCTGTGACAAATCAACCCAAACCCCT
TCTCCACCTTGCCAAGCCTTTAACCACTACCTGTCAGCTATGGCATCAATG
CGACAAGCTGAGCCAGCTGATATGCGCCCGGAAATATGGATAGCACAAG
AATTGCGGCGAATCGGcGACGAGTTTAATGCTTACTACGCGCGCCGCGTGT
TTCTGAATAATTACCAAGCCGCTGAGGACCACCCCAGAATGGTCATTCTT
AGGCTCCTGCGCTACATAGTCAGGTTGGTGTGGCGGATGCAT
(SEQ ID NO: 10)
MAKQPSDVSSECDREGRQLQPAERPPQLRPGAPTSLQTEPQGNPEGNHGGEG
DSCPHGSPQGPLAPPASPGPFATRSPLFIFMRRSSLLSRSSSGYFSFDTDRSPAP
MSCDKSTQTPSPPCQAFNHYLSAMASMRQAEPADMRPEIWIAQELRRIGDEF
NAYYARRVFLNNYQAAEDHPRMVILRLLRYIVRLVWRMH
PUMA BH3
(SEQ ID NO: 11)
GAGGAGCAGgccGCAAGAGAAATCGGAGCACAACTTCGGCGAATGGCTGA
CGACTTGAACGCACAGTATGAGCGG
(SEQ ID NO: 12)
EEQAAREIGAQLRRMADDLNAQYER
PUMA W71A
(SEQ ID NO: 13)
ATGAAATTCGGGATGGGTAGTGCCCAAGCCTGTCCCTGCCAAGTTCCGCG
CGCAGCGTCAACGACATGGGTGCCCTGCCAGATATGCGGCCCCCAACCTT
CTCTCAGCCTCGCTGAACAACATTTGGAGAGTCCTGTGCCTAGTGCTCCTG
GCGCGTTGGCGGGAGGTCCTACCCAAGCAGCTCCTGGGGTACGAGGTGAA
GAAGAGCAAgccGCCCGAGAAATAGGCGCCCAGTTGAGAAGAATGGCTGA
TGACCTCAACGCACAGTACGAAAGACGACGACAAGAGGAACAACAGCGC
CACCGGCCTTCACCATGGCGAGTTCTGTACAATCTTATTATGGGGCTTCTT
CCGCTTCCTCGGGGCCACAGAGCCCCTGAGATGGAGCCTAAC
(SEQ ID NO: 14)
MKFGMGSAQACPCQVPRAASTTWVPCQICGPQPSLSLAEQHLESPVPSAPGA
LAGGPTQAAPGVRGEEEQAAREIGAQLRRMADDLNAQYERRRQEEQQRHRP
SPWRVLYNLIMGLLPLPRGHRAPEMEPN
ICAD-degron-CAD (codon optimized, 2A sequence bold)
(SEQ ID NO: 15)
ATGGAAGTGACTGGTGATGCCGGTGTCCCAGAGAGCGGCGAGATCAGGA
CTCTTAAACCATGCCTGCTTAGGCGAAACTACAGCCGAGAGCAACATGGT
GTTGCTGCTAGTTGCTTGGAAGACCTGCGATCCAAGGCGTGTGACATCTTG
GCAATTGATAAGTCACTCACGCCGGTTACTTTGGTGCTTGCCGAAGACGG
CACAATAGTAGACGACGACGATTACTTCCTCTGCCTTCCGTCAAACACGA
AGTTTGTTGCACTTGCGAGTAATGAGAAATGGGCTTACAACAATTCAGAT
GGTGGCACCGCCTGGATTAGCCAGGAATCTTTCGACGTAGATGAAACAGA
CTCTGGGGCCGGTCTCAAATGGAAGAACGTTGCCCGGCAGCTTAAAGAAG
ATCTGTCTAGCATAATCCTGCTCTCTGAGGAGGATCTGCAGATGCTGGTGG
ATGCGCCCTGTAGTGATTTGGCCCAAGAACTTCGACAAAGTTGTGCAACT
GTACAACGGCTCCAACACACATTGCAGCAGGTGCTGGACCAGCGAGAGG
AGGTGAGGCAGAGCAAGCAACTTCTTCAACTCTACCTCCAAGCTCTTGAG
AAAGAGGGCTCATTGCTCTCAAAACAAGAAGAGTCTAAAGCAGCGTTTGG
TGAGGAAGTCGACGCTGTAGACACTGGAATTTCAAGAGAGACTAGTTCCG
ATGTTGCGCTTGCTAGTCATATTCTTACAGCTCTGAGAGAGAAGCAAGCTC
CAGAACTCTCTCTCAGCTCCCAAGACCTCGAACTTGTTACTAAAGAAGAC
CCGAAGGCGCTGGCAGTCGCTCTTAACTGGGACATCAAGAAGACAGAGA
CaGTTCAAGAGGCATGTGAACGGGAGCTGGCATTGCGCCTCCAACAAACT
CAATCCCTGCATTCCCTGCGGTCCATAAGCGCCTCAAAAGCAAGCCCTCC
AGGGGATCTGCAGAATCCTAAGAGGGCGCGGCAAGATCCCACGggcagtggct
cgggctcggggtccggcggaTTCAATGTCTTAATGGTTCATAAGCGAAGCCATACTG
GTGAACGCCCATTGCAGTGCGAAATATGCGGCTTTACCTGCCGCCAGAAA
GGTAACCTCCTCCGCCACATTAAACTGCACACAGGGGAAAAACCTTTTAA
GTGTCACCTCTGCAACTATGCATGCCAAAGAAGAGATGCGCTCTCCGGAG
GCGGCGGAGAGGGCAGAGGAAGTCTTCTAACATGCGGTGACGTGGAGGA
GAATCCCGGCCCTAGGATGCTTCAGAAACCCAAGAGTGTGAAGTTGCGGG
CCTTGCGCTCACCTAGAAAGTTTGGAGTAGCTGGACGAAGTTGTCAGGAG
GTGCTTCGGAAAGGCTGCCTGCGATTCCAGCTGCCCGAAAGAGGTTCTCG
GTTGTGTTTGTATGAGGATGGAACCGAACTGACAGAGGACTATTTTCCCTC
CGTACCTGACAATGCGGAGTTGGTACTTTTGACCCTTGGTCAAGCTTGGCA
GGGATATGTCTCTGATATAAGGCGCTTCCTCTCAGCTTTCCACGAACCCCA
GGTGGGGCTCATACAAGCAGCGCAGCAGCTGCTGTGCGACGAGCAAGCA
CCCCAACGACAACGACTGTTGGCGGACTTGTTGCATAACGTATCCCAAAA
TATTGCGGCGGAGACACGCGCCGAGGACCCTCCATGGTTCGAGGGACTTG
AAAGTCGCTTTCAGTCCAAAAGCGGATATCTTCGATACTCATGTGAATCTC
GCATTAGAAGCTACCTTAGAGAAGTCTCTAGTTATCCGAGTACTGTCGGC
GCAGAGGCACAAGAGGAGTTCTTGAGAGTCTTGGGCTCCATGTGTCAACG
GTTGCGGTCCATGCAATACAATGGCAGCTATTTTGACCGGGGCGCTAAAG
GTGGCTCTCGGCTTTGCACACCCGAGGGCTGGTTCTCTTGCCAGGGCCCCT
TCGACATGGACTCTTGCCTTTCACGGCACTCTATCAATCCATACTCCAATC
GGGAATCCAGGATTCTCTTCAGCACCTGGAATCTCGATCATATAATAGAA
AAGAAACGGACTATTATACCAACTCTCGTAGAAGCCATAAAGGAACAGG
ATGGGCGGGAAGTAGATTGGGAATATTTCTATGGCCTCCTTTTCACTTCCG
AAAACTTGAAACTGGTGCATATAGTTTGCCATAAGAAGACCACCCATAAG
TTGAACTGCGATCCCTCAAGAATATATAAACCTCAGACCAGGCTCAAGCG
AAAGCAGCCTGTCCGAAAACGACAG
(SEQ ID NO: 16)
MEVTGDAGVPESGEIRTLKPCLLRRNYSREQHGVAASCLEDLRSKACDILAID
KSLTPVTLVLAEDGTIVDDDDYFLCLPSNTKFVALASNEKWAYNNSDGGTA
WISQESFDVDETDSGAGLKWKNVARQLKEDLSSIILLSEEDLQMLVDAPCSDL
AQELRQSCATVQRLQHTLQQVLDQREEVRQSKQLLQLYLQALEKEGSLLSK
QEESKAAFGEEVDAVDTGISRETSSDVALASHILTALREKQAPELSLSSQDLEL
VTKEDPKALAVALNWDIKKTETVQEACERELALRLQQTQSLHSLRSISASKAS
PPGDLQNPKRARQDPTGSGSGSGSGGFNVLMVHKRSHTGERPLQCEICGFTC
RQKGNLLRHIKLHTGEKPFKCHLCNYACQRRDALSGGGGEGRGSLLTCGD
VEENPGPRMLQKPKSVKLRALRSPRKFGVAGRSCQEVLRKGCLRFQLPERGS
RLCLYEDGTELTEDYFPSVPDNAELVLLTLGQAWQGYVSDIRRFLSAFHEPQ
VGLIQAAQQLLCDEQAPQRQRLLADLLHNVSQNIAAETRAEDPPWFEGLESR
FQSKSGYLRYSCESRIRSYLREVSSYPSTVGAEAQEEFLRVLGSMCQRLRSMQ
YNGSYFDRGAKGGSRLCTPEGWFSCQGPFDMDSCLSRHSINPYSNRESRILFS
TWNLDHIIEKKRTIIPTLVEAIKEQDGREVDWEYFYGLLFTSENLKLVHIVCHK
KTTHKLNCDPSRIYKPQTRLKRKQPVRKRQ
ICAD-degron-CAD
(SEQ ID NO: 17)
ATGGAAGTGACTGGTGATGCCGGTGTCCCAGAGAGCGGCGAGATCAGGA
CTCTTAAACCATGCCTGCTTAGGCGAAACTACAGCCGAGAGCAACATGGT
GTTGCTGCTAGTTGCTTGGAAGACCTGCGATCCAAGGCGTGTGACATCTTG
GCAATTGATAAGTCACTCACGCCGGTTACTTTGGTGCTTGCCGAAGACGG
CACAATAGTAGACGACGACGATTACTTCCTCTGCCTTCCGTCAAACACGA
AGTTTGTTGCACTTGCGAGTAATGAGAAATGGGCTTACAACAATTCAGAT
GGTGGCACCGCCTGGATTAGCCAGGAATCTTTCGACGTAGATGAAACAGA
CTCTGGGGCCGGTCTCAAATGGAAGAACGTTGCCCGGCAGCTTAAAGAAG
ATCTGTCTAGCATAATCCTGCTCTCTGAGGAGGATCTGCAGATGCTGGTGG
ATGCGCCCTGTAGTGATTTGGCCCAAGAACTTCGACAAAGTTGTGCAACT
GTACAACGGCTCCAACACACATTGCAGCAGGTGCTGGACCAGCGAGAGG
AGGTGAGGCAGAGCAAGCAACTTCTTCAACTCTACCTCCAAGCTCTTGAG
AAAGAGGGCTCATTGCTCTCAAAACAAGAAGAGTCTAAAGCAGCGTTTGG
TGAGGAAGTCGACGCTGTAGACACTGGAATTTCAAGAGAGACTAGTTCCG
ATGTTGCGCTTGCTAGTCATATTCTTACAGCTCTGAGAGAGAAGCAAGCTC
CAGAACTCTCTCTCAGCTCCCAAGACCTCGAACTTGTTACTAAAGAAGAC
CCGAAGGCGCTGGCAGTCGCTCTTAACTGGGACATCAAGAAGACAGAGA
CaGTTCAAGAGGCATGTGAACGGGAGCTGGCATTGCGCCTCCAACAAACT
CAATCCCTGCATTCCCTGCGGTCCATAAGCGCCTCAAAAGCAAGCCCTCC
AGGGGATCTGCAGAATCCTAAGAGGGCGCGGCAAGATCCCACGggcagtggct
cgggctcggggtccggcggaTTCAATGTCTTAATGGTTCATAAGCGAAGCCATACTG
GTGAACGCCCATTGCAGTGCGAAATATGCGGCTTTACCTGCCGCCAGAAA
GGTAACCTCCTCCGCCACATTAAACTGCACACAGGGGAAAAACCTTTTAA
GTGTCACCTCTGCAACTATGCATGCCAAAGAAGAGATGCGCTCTCCGGAG
GCGGCGGAGAGGGCAGAGGAAGTCTTCTAACATGCGGTGACGTGGAGGA
GAATCCCGGCCCTAGGATGCTCCAGAAGCCCAAGAGCGTGAAGCTGCGG
GCCCTGCGCAGCCCGAGGAAGTTCGGCGTGGCTGGCCGGAGCTGCCAGGA
GGTGCTGCGCAAGGGCTGTCTCCGCTTCCAGCTCCCTGAGCGCGGTTCCCG
GCTGTGCCTGTACGAGGATGGCACGGAGCTGACGGAAGATTACTTCCCCA
GTGTTCCCGACAACGCCGAGCTGGTGCTGCTCACCTTGGGCCAGGCCTGG
CAGGGCTATGTGAGCGACATCAGGCGCTTCCTCAGTGCATTTCACGAGCC
ACAGGTGGGGCTCATCCAGGCCGCCCAGCAGCTGCTGTGTGATGAGCAGG
CCCCACAGAGGCAGAGGCTGCTGGCTGACCTCCTGCACAACGTCAGCCAG
AACATCGCGGCCGAGACCCGGGCTGAGGACCCGCCGTGGTTTGAAGGCTT
GGAGTCCCGATTTCAGAGCAAGTCTGGCTATCTGAGATACAGCTGTGAGA
GCCGGATCCGGAGTTACCTGAGGGAGGTGAGCTCCTACCCCTCCACGGTG
GGTGCGGAGGCTCAGGAGGAATTCCTGCGGGTCCTCGGCTCCATGTGCCA
GAGGCTCCGGTCCATGCAGTACAATGGCAGCTACTTCGACAGAGGAGCCA
AGGGCGGCAGCCGCCTCTGCACACCGGAAGGCTGGTTCTCCTGCCAGGGT
CCCTTTGACATGGACAGCTGCTTATCAAGACACTCCATCAACCCCTACAGT
AACAGGGAGAGCAGGATCCTCTTCAGCACCTGGAACCTGGATCACATAAT
AGAAAAGAAACGCACCATCATTCCTACACTGGTGGAAGCAATTAAGGAA
CAAGATGGAAGAGAAGTGGACTGGGAGTATTTTTATGGCCTGCTTTTTAC
CTCAGAGAACCTAAAACTAGTGCACATTGTCTGCCATAAGAAAACCACCC
ACAAGCTCAACTGTGACCCAAGCAGAATCTACAAACCCCAGACAAGGTTG
AAGCGGAAGCAGCCTGTGCGGAAACGCCAG
(SEQ ID NO: 18)
MEVTGDAGVPESGEIRTLKPCLLRRNYSREQHGVAASCLEDLRSKACDILAID
KSLTPVTLVLAEDGTIVDDDDYFLCLPSNTKFVALASNEKWAYNNSDGGTA
WISQESFDVDETDSGAGLKWKNVARQLKEDLSSIILLSEEDLQMLVDAPCSDL
AQELRQSCATVQRLQHTLQQVLDQREEVRQSKQLLQLYLQALEKEGSLLSK
QEESKAAFGEEVDAVDTGISRETSSDVALASHILTALREKQAPELSLSSQDLEL
VTKEDPKALAVALNWDIKKTETVQEACERELALRLQQTQSLHSLRSISASKAS
PPGDLQNPKRARQDPTGSGSGSGSGGFNVLMVHKRSHTGERPLQCEICGFTC
RQKGNLLRHIKLHTGEKPFKCHLCNYACQRRDALSGGGGEGRGSLLTCGDV
EENPGPRMLQKPKSVKLRALRSPRKFGVAGRSCQEVLRKGCLRFQLPERGSR
LCLYEDGTELTEDYFPSVPDNAELVLLTLGQAWQGYVSDIRRFLSAFHEPQV
GLIQAAQQLLCDEQAPQRQRLLADLLHNVSQNIAAETRAEDPPWFEGLESRF
QSKSGYLRYSCESRIRSYLREVSSYPSTVGAEAQEEFLRVLGSMCQRLRSMQY
NGSYFDRGAKGGSRLCTPEGWFSCQGPFDMDSCLSRHSINPYSNRESRILFST
WNLDHIIEKKRTIIPTLVEAIKEQDGREVDWEYFYGLLFTSENLKLVHIVCHKK
TTHKLNCDPSRIYKPQTRLKRKQPVRKRQ
ICAD-degron-CADp.R11W
(SEQ ID NO: 19)
ATGGAAGTGACTGGTGATGCCGGTGTCCCAGAGAGCGGCGAGATCAGGA
CTCTTAAACCATGCCTGCTTAGGCGAAACTACAGCCGAGAGCAACATGGT
GTTGCTGCTAGTTGCTTGGAAGACCTGCGATCCAAGGCGTGTGACATCTTG
GCAATTGATAAGTCACTCACGCCGGTTACTTTGGTGCTTGCCGAAGACGG
CACAATAGTAGACGACGACGATTACTTCCTCTGCCTTCCGTCAAACACGA
AGTTTGTTGCACTTGCGAGTAATGAGAAATGGGCTTACAACAATTCAGAT
GGTGGCACCGCCTGGATTAGCCAGGAATCTTTCGACGTAGATGAAACAGA
CTCTGGGGCCGGTCTCAAATGGAAGAACGTTGCCCGGCAGCTTAAAGAAG
ATCTGTCTAGCATAATCCTGCTCTCTGAGGAGGATCTGCAGATGCTGGTGG
ATGCGCCCTGTAGTGATTTGGCCCAAGAACTTCGACAAAGTTGTGCAACT
GTACAACGGCTCCAACACACATTGCAGCAGGTGCTGGACCAGCGAGAGG
AGGTGAGGCAGAGCAAGCAACTTCTTCAACTCTACCTCCAAGCTCTTGAG
AAAGAGGGCTCATTGCTCTCAAAACAAGAAGAGTCTAAAGCAGCGTTTGG
TGAGGAAGTCGACGCTGTAGACACTGGAATTTCAAGAGAGACTAGTTCCG
ATGTTGCGCTTGCTAGTCATATTCTTACAGCTCTGAGAGAGAAGCAAGCTC
CAGAACTCTCTCTCAGCTCCCAAGACCTCGAACTTGTTACTAAAGAAGAC
CCGAAGGCGCTGGCAGTCGCTCTTAACTGGGACATCAAGAAGACAGAGA
CaGTTCAAGAGGCATGTGAACGGGAGCTGGCATTGCGCCTCCAACAAACT
CAATCCCTGCATTCCCTGCGGTCCATAAGCGCCTCAAAAGCAAGCCCTCC
AGGGGATCTGCAGAATCCTAAGAGGGCGCGGCAAGATCCCACGggcagtggct
cgggctcggggtccggcggaTTCAATGTCTTAATGGTTCATAAGCGAAGCCATACTG
GTGAACGCCCATTGCAGTGCGAAATATGCGGCTTTACCTGCCGCCAGAAA
GGTAACCTCCTCCGCCACATTAAACTGCACACAGGGGAAAAACCTTTTAA
GTGTCACCTCTGCAACTATGCATGCCAAAGAAGAGATGCGCTCTCCGGAG
GCGGCGGAGAGGGCAGAGGAAGTCTTCTAACATGCGGTGACGTGGAGGA
GAATCCCGGCCCTAGGATGCTCCAGAAGCCCAAGAGCGTGAAGCTGtGGG
CCCTGCGCAGCCCGAGGAAGTTCGGCGTGGCTGGCCGGAGCTGCCAGGAG
GTGCTGCGCAAGGGCTGTCTCCGCTTCCAGCTCCCTGAGCGCGGTTCCCGG
CTGTGCCTGTACGAGGATGGCACGGAGCTGACGGAAGATTACTTCCCCAG
TGTTCCCGACAACGCCGAGCTGGTGCTGCTCACCTTGGGCCAGGCCTGGC
AGGGCTATGTGAGCGACATCAGGCGCTTCCTCAGTGCATTTCACGAGCCA
CAGGTGGGGCTCATCCAGGCCGCCCAGCAGCTGCTGTGTGATGAGCAGGC
CCCACAGAGGCAGAGGCTGCTGGCTGACCTCCTGCACAACGTCAGCCAGA
ACATCGCGGCCGAGACCCGGGCTGAGGACCCGCCGTGGTTTGAAGGCTTG
GAGTCCCGATTTCAGAGCAAGTCTGGCTATCTGAGATACAGCTGTGAGAG
CCGGATCCGGAGTTACCTGAGGGAGGTGAGCTCCTACCCCTCCACGGTGG
GTGCGGAGGCTCAGGAGGAATTCCTGCGGGTCCTCGGCTCCATGTGCCAG
AGGCTCCGGTCCATGCAGTACAATGGCAGCTACTTCGACAGAGGAGCCAA
GGGCGGCAGCCGCCTCTGCACACCGGAAGGCTGGTTCTCCTGCCAGGGTC
CCTTTGACATGGACAGCTGCTTATCAAGACACTCCATCAACCCCTACAGTA
ACAGGGAGAGCAGGATCCTCTTCAGCACCTGGAACCTGGATCACATAATA
GAAAAGAAACGCACCATCATTCCTACACTGGTGGAAGCAATTAAGGAAC
AAGATGGAAGAGAAGTGGACTGGGAGTATTTTTATGGCCTGCTTTTTACCT
CAGAGAACCTAAAACTAGTGCACATTGTCTGCCATAAGAAAACCACCCAC
AAGCTCAACTGTGACCCAAGCAGAATCTACAAACCCCAGACAAGGTTGAA
GCGGAAGCAGCCTGTGCGGAAACGCCAG
(SEQ ID NO: 20)
MEVTGDAGVPESGEIRTLKPCLLRRNYSREQHGVAASCLEDLRSKACDILAID
KSLTPVTLVLAEDGTIVDDDDYFLCLPSNTKFVALASNEKWAYNNSDGGTA
WISQESFDVDETDSGAGLKWKNVARQLKEDLSSIILLSEEDLQMLVDAPCSDL
AQELRQSCATVQRLQHTLQQVLDQREEVRQSKQLLQLYLQALEKEGSLLSK
QEESKAAFGEEVDAVDTGISRETSSDVALASHILTALREKQAPELSLSSQDLEL
VTKEDPKALAVALNWDIKKTETVQEACERELALRLQQTQSLHSLRSISASKAS
PPGDLQNPKRARQDPTGSGSGSGSGGFNVLMVHKRSHTGERPLQCEICGFTC
RQKGNLLRHIKLHTGEKPFKCHLCNYACQRRDALSGGGGEGRGSLLTCGDV
EENPGPRMLQKPKSVKLWALRSPRKFGVAGRSCQEVLRKGCLRFQLPERGSR
LCLYEDGTELTEDYFPSVPDNAELVLLTLGQAWQGYVSDIRRFLSAFHEPQV
GLIQAAQQLLCDEQAPQRQRLLADLLHNVSQNIAAETRAEDPPWFEGLESRF
QSKSGYLRYSCESRIRSYLREVSSYPSTVGAEAQEEFLRVLGSMCQRLRSMQY
NGSYFDRGAKGGSRLCTPEGWFSCQGPFDMDSCLSRHSINPYSNRESRILFST
WNLDHIIEKKRTIIPTLVEAIKEQDGREVDWEYFYGLLFTSENLKLVHIVCHKK
TTHKLNCDPSRIYKPQTRLKRKQPVRKRQ
GFP-ICAD-degron (Gly-Ser linker in lower case in DNA sequence)
(SEQ ID NO: 21)
ATGGTGAGCAAGGGCGAGGAGCTGTTCACCGGGGTGGTGCCCATCCTGGT
CGAGCTGGACGGCGACGTAAACGGCCACAAGTTCAGCGTGTCCGGCGAG
GGCGAGGGCGATGCCACCTACGGCAAGCTGACCCTGAAGTTCATCTGCAC
CACCGGCAAGCTGCCCGTGCCCTGGCCCACCCTCGTGACCACCCTGACCT
ACGGCGTGCAGTGCTTCAGCCGCTACCCCGACCACATGAAGCAGCACGAC
TTCTTCAAGTCCGCCATGCCCGAAGGCTACGTCCAGGAGCGCACCATCTTC
TTCAAGGACGACGGCAACTACAAGACCCGCGCCGAGGTGAAGTTCGAGG
GCGACACCCTGGTGAACCGCATCGAGCTGAAGGGCATCGACTTCAAGGAG
GACGGCAACATCCTGGGGCACAAGCTGGAGTACAACTACAACAGCCACA
ACGTCTATATCATGGCCGACAAGCAGAAGAACGGCATCAAGGTGAACTTC
AAGATCCGCCACAACATCGAGGACGGCAGCGTGCAGCTCGCCGACCACTA
CCAGCAGAACACCCCCATCGGCGACGGCCCCGTGCTGCTGCCCGACAACC
ACTACCTGAGCACCCAGTCCGCCCTGAGCAAAGACCCCAACGAGAAGCGC
GATCACATGGTCCTGCTGGAGTTCGTGACCGCCGCCGGGATCACTCTCGG
CATGGACGAGCTGTACAAGGGCGGCGGCGGCTCCGGCGGCGGCGGCTCC
ATGGAAGTGACTGGTGATGCCGGTGTCCCAGAGAGCGGCGAGATCAGGA
CTCTTAAACCATGCCTGCTTAGGCGAAACTACAGCCGAGAGCAACATGGT
GTTGCTGCTAGTTGCTTGGAAGACCTGCGATCCAAGGCGTGTGACATCTTG
GCAATTGATAAGTCACTCACGCCGGTTACTTTGGTGCTTGCCGAAGACGG
CACAATAGTAGACGACGACGATTACTTCCTCTGCCTTCCGTCAAACACGA
AGTTTGTTGCACTTGCGAGTAATGAGAAATGGGCTTACAACAATTCAGAT
GGTGGCACCGCCTGGATTAGCCAGGAATCTTTCGACGTAGATGAAACAGA
CTCTGGGGCCGGTCTCAAATGGAAGAACGTTGCCCGGCAGCTTAAAGAAG
ATCTGTCTAGCATAATCCTGCTCTCTGAGGAGGATCTGCAGATGCTGGTGG
ATGCGCCCTGTAGTGATTTGGCCCAAGAACTTCGACAAAGTTGTGCAACT
GTACAACGGCTCCAACACACATTGCAGCAGGTGCTGGACCAGCGAGAGG
AGGTGAGGCAGAGCAAGCAACTTCTTCAACTCTACCTCCAAGCTCTTGAG
AAAGAGGGCTCATTGCTCTCAAAACAAGAAGAGTCTAAAGCAGCGTTTGG
TGAGGAAGTCGACGCTGTAGACACTGGAATTTCAAGAGAGACTAGTTCCG
ATGTTGCGCTTGCTAGTCATATTCTTACAGCTCTGAGAGAGAAGCAAGCTC
CAGAACTCTCTCTCAGCTCCCAAGACCTCGAACTTGTTACTAAAGAAGAC
CCGAAGGCGCTGGCAGTCGCTCTTAACTGGGACATCAAGAAGACAGAGA
CaGTTCAAGAGGCATGTGAACGGGAGCTGGCATTGCGCCTCCAACAAACT
CAATCCCTGCATTCCCTGCGGTCCATAAGCGCCTCAAAAGCAAGCCCTCC
AGGGGATCTGCAGAATCCTAAGAGGGCGCGGCAAGATCCCACGggcagtggct
cgggctcggggtccggcggaTTCAATGTCTTAATGGTTCATAAGCGAAGCCATACTG
GTGAACGCCCATTGCAGTGCGAAATATGCGGCTTTACCTGCCGCCAGAAA
GGTAACCTCCTCCGCCACATTAAACTGCACACAGGGGAAAAACCTTTTAA
GTGTCACCTCTGCAACTATGCATGCCAAAGAAGAGATGCGCTC
(SEQ ID NO: 22)
MVSKGEELFTGVVPILVELDGDVNGHKFSVSGEGEGDATYGKLTLKFICTTG
KLPVPWPTLVTTLTYGVQCFSRYPDHMKQHDFFKSAMPEGYVQERTIFFKDD
GNYKTRAEVKFEGDTLVNRIELKGIDFKEDGNILGHKLEYNYNSHNVYIMAD
KQKNGIKVNFKIRHNIEDGSVQLADHYQQNTPIGDGPVLLPDNHYLSTQSALS
KDPNEKRDHMVLLEFVTAAGITLGMDELYKGGGGSGGGGSMEVTGDAGVP
ESGEIRTLKPCLLRRNYSREQHGVAASCLEDLRSKACDILAIDKSLTPVTLVLA
EDGTIVDDDDYFLCLPSNTKFVALASNEKWAYNNSDGGTAWISQESFDVDET
DSGAGLKWKNVARQLKEDLSSIILLSEEDLQMLVDAPCSDLAQELRQSCATV
QRLQHTLQQVLDQREEVRQSKQLLQLYLQALEKEGSLLSKQEESKAAFGEEV
DAVDTGISRETSSDVALASHILTALREKQAPELSLSSQDLELVTKEDPKALAV
ALNWDIKKTETVQEACERELALRLQQTQSLHSLRSISASKASPPGDLQNPKRA
RQDPTGSGSGSGSGGFNVLMVHKRSHTGERPLQCEICGFTCRQKGNLLRHIK
LHTGEKPFKCHLCNYACQRRDAL
ICAD-degron-CADp.R11A
(SEQ ID NO: 23)
ATGGAAGTGACTGGTGATGCCGGTGTCCCAGAGAGCGGCGAGATCAGGA
CTCTTAAACCATGCCTGCTTAGGCGAAACTACAGCCGAGAGCAACATGGT
GTTGCTGCTAGTTGCTTGGAAGACCTGCGATCCAAGGCGTGTGACATCTTG
GCAATTGATAAGTCACTCACGCCGGTTACTTTGGTGCTTGCCGAAGACGG
CACAATAGTAGACGACGACGATTACTTCCTCTGCCTTCCGTCAAACACGA
AGTTTGTTGCACTTGCGAGTAATGAGAAATGGGCTTACAACAATTCAGAT
GGTGGCACCGCCTGGATTAGCCAGGAATCTTTCGACGTAGATGAAACAGA
CTCTGGGGCCGGTCTCAAATGGAAGAACGTTGCCCGGCAGCTTAAAGAAG
ATCTGTCTAGCATAATCCTGCTCTCTGAGGAGGATCTGCAGATGCTGGTGG
ATGCGCCCTGTAGTGATTTGGCCCAAGAACTTCGACAAAGTTGTGCAACT
GTACAACGGCTCCAACACACATTGCAGCAGGTGCTGGACCAGCGAGAGG
AGGTGAGGCAGAGCAAGCAACTTCTTCAACTCTACCTCCAAGCTCTTGAG
AAAGAGGGCTCATTGCTCTCAAAACAAGAAGAGTCTAAAGCAGCGTTTGG
TGAGGAAGTCGACGCTGTAGACACTGGAATTTCAAGAGAGACTAGTTCCG
ATGTTGCGCTTGCTAGTCATATTCTTACAGCTCTGAGAGAGAAGCAAGCTC
CAGAACTCTCTCTCAGCTCCCAAGACCTCGAACTTGTTACTAAAGAAGAC
CCGAAGGCGCTGGCAGTCGCTCTTAACTGGGACATCAAGAAGACAGAGA
CaGTTCAAGAGGCATGTGAACGGGAGCTGGCATTGCGCCTCCAACAAACT
CAATCCCTGCATTCCCTGCGGTCCATAAGCGCCTCAAAAGCAAGCCCTCC
AGGGGATCTGCAGAATCCTAAGAGGGCGCGGCAAGATCCCACGggcagtggct
cgggctcggggtccggcggaTTCAATGTCTTAATGGTTCATAAGCGAAGCCATACTG
GTGAACGCCCATTGCAGTGCGAAATATGCGGCTTTACCTGCCGCCAGAAA
GGTAACCTCCTCCGCCACATTAAACTGCACACAGGGGAAAAACCTTTTAA
GTGTCACCTCTGCAACTATGCATGCCAAAGAAGAGATGCGCTCTCCGGAG
GCGGCGGAGAGGGCAGAGGAAGTCTTCTAACATGCGGTGACGTGGAGGA
GAATCCCGGCCCTAGGATGCTCCAGAAGCCCAAGAGCGTGAAGCTGgccGC
CCTGCGCAGCCCGAGGAAGTTCGGCGTGGCTGGCCGGAGCTGCCAGGAG
GTGCTGCGCAAGGGCTGTCTCCGCTTCCAGCTCCCTGAGCGCGGTTCCCGG
CTGTGCCTGtacGAGGATGGCACGGAGCTGACGGAAGATTACTTCCCCAGT
GTTCCCGACAACGCCGAGCTGGTGCTGCTCACCTTGGGCCAGGCCTGGCA
GGGCTATGTGAGCGACATCAGGCGCTTCCTCAGTGCATTTCACGAGCCAC
AGGTGGGGCTCATCCAGGCCGCCCAGCAGCTGCTGTGTGATGAGCAGGCC
CCACAGAGGCAGAGGCTGCTGGCTGACCTCCTGCACAACGTCAGCCAGAA
CATCGCGGCCGAGACCCGGGCTGAGGACCCGCCGTGGTTTGAAGGCTTGG
AGTCCCGATTTCAGAGCAAGTCTGGCTATCTGAGATACAGCTGTGAGAGC
CGGATCCGGAGTTACCTGAGGGAGGTGAGCTCCTACCCCTCCACGGTGGG
TGCGGAGGCTCAGGAGGAATTCCTGCGGGTCCTCGGCTCCATGTGCCAGA
GGCTCCGGTCCATGCAGTACAATGGCAGCTACTTCGACAGAGGAGCCAAG
GGCGGCAGCCGCCTCTGCACACCGGAAGGCTGGTTCTCCTGCCAGGGTCC
CTTTGACATGGACAGCTGCTTATCAAGACACTCCATCAACCCCTACAGTA
ACAGGGAGAGCAGGATCCTCTTCAGCACCTGGAACCTGGATCACATAATA
GAAAAGAAACGCACCATCATTCCTACACTGGTGGAAGCAATTAAGGAAC
AAGATGGAAGAGAAGTGGACTGGGAGTATTTTTATGGCCTGCTTTTTACCT
CAGAGAACCTAAAACTAGTGCACATTGTCTGCCATAAGAAAACCACCCAC
AAGCTCAACTGTGACCCAAGCAGAATCTACAAACCCCAGACAAGGTTGAA
GCGGAAGCAGCCTGTGCGGAAACGCCAG
(SEQ ID NO: 24)
MEVTGDAGVPESGEIRTLKPCLLRRNYSREQHGVAASCLEDLRSKACDILAID
KSLTPVTLVLAEDGTIVDDDDYFLCLPSNTKFVALASNEKWAYNNSDGGTA
WISQESFDVDETDSGAGLKWKNVARQLKEDLSSIILLSEEDLQMLVDAPCSDL
AQELRQSCATVQRLQHTLQQVLDQREEVRQSKQLLQLYLQALEKEGSLLSK
QEESKAAFGEEVDAVDTGISRETSSDVALASHILTALREKQAPELSLSSQDLEL
VTKEDPKALAVALNWDIKKTETVQEACERELALRLQQTQSLHSLRSISASKAS
PPGDLQNPKRARQDPTGSGSGSGSGGFNVLMVHKRSHTGERPLQCEICGFTC
RQKGNLLRHIKLHTGEKPFKCHLCNYACQRRDALSGGGGEGRGSLLTCGDV
EENPGPRMLQKPKSVKLAALRSPRKFGVAGRSCQEVLRKGCLRFQLPERGSR
LCLYEDGTELTEDYFPSVPDNAELVLLTLGQAWQGYVSDIRRFLSAFHEPQV
GLIQAAQQLLCDEQAPQRQRLLADLLHNVSQNIAAETRAEDPPWFEGLESRF
QSKSGYLRYSCESRIRSYLREVSSYPSTVGAEAQEEFLRVLGSMCQRLRSMQY
NGSYFDRGAKGGSRLCTPEGWFSCQGPFDMDSCLSRHSINPYSNRESRILFST
WNLDHIIEKKRTIIPTLVEAIKEQDGREVDWEYFYGLLFTSENLKLVHIVCHKK
TTHKLNCDPSRIYKPQTRLKRKQPVRKRQ
ICAD-degron-CADp.Y49W
(SEQ ID NO: 25)
ATGGAAGTGACTGGTGATGCCGGTGTCCCAGAGAGCGGCGAGATCAGGA
CTCTTAAACCATGCCTGCTTAGGCGAAACTACAGCCGAGAGCAACATGGT
GTTGCTGCTAGTTGCTTGGAAGACCTGCGATCCAAGGCGTGTGACATCTTG
GCAATTGATAAGTCACTCACGCCGGTTACTTTGGTGCTTGCCGAAGACGG
CACAATAGTAGACGACGACGATTACTTCCTCTGCCTTCCGTCAAACACGA
AGTTTGTTGCACTTGCGAGTAATGAGAAATGGGCTTACAACAATTCAGAT
GGTGGCACCGCCTGGATTAGCCAGGAATCTTTCGACGTAGATGAAACAGA
CTCTGGGGCCGGTCTCAAATGGAAGAACGTTGCCCGGCAGCTTAAAGAAG
ATCTGTCTAGCATAATCCTGCTCTCTGAGGAGGATCTGCAGATGCTGGTGG
ATGCGCCCTGTAGTGATTTGGCCCAAGAACTTCGACAAAGTTGTGCAACT
GTACAACGGCTCCAACACACATTGCAGCAGGTGCTGGACCAGCGAGAGG
AGGTGAGGCAGAGCAAGCAACTTCTTCAACTCTACCTCCAAGCTCTTGAG
AAAGAGGGCTCATTGCTCTCAAAACAAGAAGAGTCTAAAGCAGCGTTTGG
TGAGGAAGTCGACGCTGTAGACACTGGAATTTCAAGAGAGACTAGTTCCG
ATGTTGCGCTTGCTAGTCATATTCTTACAGCTCTGAGAGAGAAGCAAGCTC
CAGAACTCTCTCTCAGCTCCCAAGACCTCGAACTTGTTACTAAAGAAGAC
CCGAAGGCGCTGGCAGTCGCTCTTAACTGGGACATCAAGAAGACAGAGA
CaGTTCAAGAGGCATGTGAACGGGAGCTGGCATTGCGCCTCCAACAAACT
CAATCCCTGCATTCCCTGCGGTCCATAAGCGCCTCAAAAGCAAGCCCTCC
AGGGGATCTGCAGAATCCTAAGAGGGCGCGGCAAGATCCCACGggcagtggct
cgggctcggggtccggcggaTTCAATGTCTTAATGGTTCATAAGCGAAGCCATACTG
GTGAACGCCCATTGCAGTGCGAAATATGCGGCTTTACCTGCCGCCAGAAA
GGTAACCTCCTCCGCCACATTAAACTGCACACAGGGGAAAAACCTTTTAA
GTGTCACCTCTGCAACTATGCATGCCAAAGAAGAGATGCGCTCTCCGGAG
GCGGCGGAGAGGGCAGAGGAAGTCTTCTAACATGCGGTGACGTGGAGGA
GAATCCCGGCCCTAGGATGCTCCAGAAGCCCAAGAGCGTGAAGCTGaGGG
CCCTGCGCAGCCCGAGGAAGTTCGGCGTGGCTGGCCGGAGCTGCCAGGAG
GTGCTGCGCAAGGGCTGTCTCCGCTTCCAGCTCCCTGAGCGCGGTTCCCGG
CTGTGCCTGtggGAGGATGGCACGGAGCTGACGGAAGATTACTTCCCCAGT
GTTCCCGACAACGCCGAGCTGGTGCTGCTCACCTTGGGCCAGGCCTGGCA
GGGCTATGTGAGCGACATCAGGCGCTTCCTCAGTGCATTTCACGAGCCAC
AGGTGGGGCTCATCCAGGCCGCCCAGCAGCTGCTGTGTGATGAGCAGGCC
CCACAGAGGCAGAGGCTGCTGGCTGACCTCCTGCACAACGTCAGCCAGAA
CATCGCGGCCGAGACCCGGGCTGAGGACCCGCCGTGGTTTGAAGGCTTGG
AGTCCCGATTTCAGAGCAAGTCTGGCTATCTGAGATACAGCTGTGAGAGC
CGGATCCGGAGTTACCTGAGGGAGGTGAGCTCCTACCCCTCCACGGTGGG
TGCGGAGGCTCAGGAGGAATTCCTGCGGGTCCTCGGCTCCATGTGCCAGA
GGCTCCGGTCCATGCAGTACAATGGCAGCTACTTCGACAGAGGAGCCAAG
GGCGGCAGCCGCCTCTGCACACCGGAAGGCTGGTTCTCCTGCCAGGGTCC
CTTTGACATGGACAGCTGCTTATCAAGACACTCCATCAACCCCTACAGTA
ACAGGGAGAGCAGGATCCTCTTCAGCACCTGGAACCTGGATCACATAATA
GAAAAGAAACGCACCATCATTCCTACACTGGTGGAAGCAATTAAGGAAC
AAGATGGAAGAGAAGTGGACTGGGAGTATTTTTATGGCCTGCTTTTTACCT
CAGAGAACCTAAAACTAGTGCACATTGTCTGCCATAAGAAAACCACCCAC
AAGCTCAACTGTGACCCAAGCAGAATCTACAAACCCCAGACAAGGTTGAA
GCGGAAGCAGCCTGTGCGGAAACGCCAG
(SEQ ID NO: 26)
MEVTGDAGVPESGEIRTLKPCLLRRNYSREQHGVAASCLEDLRSKACDILAID
KSLTPVTLVLAEDGTIVDDDDYFLCLPSNTKFVALASNEKWAYNNSDGGTA
WISQESFDVDETDSGAGLKWKNVARQLKEDLSSIILLSEEDLQMLVDAPCSDL
AQELRQSCATVQRLQHTLQQVLDQREEVRQSKQLLQLYLQALEKEGSLLSK
QEESKAAFGEEVDAVDTGISRETSSDVALASHILTALREKQAPELSLSSQDLEL
VTKEDPKALAVALNWDIKKTETVQEACERELALRLQQTQSLHSLRSISASKAS
PPGDLQNPKRARQDPTGSGSGSGSGGFNVLMVHKRSHTGERPLQCEICGFTC
RQKGNLLRHIKLHTGEKPFKCHLCNYACQRRDALSGGGGEGRGSLLTCGDV
EENPGPRMLQKPKSVKLRALRSPRKFGVAGRSCQEVLRKGCLRFQLPERGSR
LCLWEDGTELTEDYFPSVPDNAELVLLTLGQAWQGYVSDIRRFLSAFHEPQV
GLIQAAQQLLCDEQAPQRQRLLADLLHNVSQNIAAETRAEDPPWFEGLESRF
QSKSGYLRYSCESRIRSYLREVSSYPSTVGAEAQEEFLRVLGSMCQRLRSMQY
NGSYFDRGAKGGSRLCTPEGWFSCQGPFDMDSCLSRHSINPYSNRESRILFST
WNLDHIIEKKRTIIPTLVEAIKEQDGREVDWEYFYGLLFTSENLKLVHIVCHKK
TTHKLNCDPSRIYKPQTRLKRKQPVRKRQ
ICAD-degron-CADp.R11W/K18A
(SEQ ID NO: 27)
ATGGAAGTGACTGGTGATGCCGGTGTCCCAGAGAGCGGCGAGATCAGGA
CTCTTAAACCATGCCTGCTTAGGCGAAACTACAGCCGAGAGCAACATGGT
GTTGCTGCTAGTTGCTTGGAAGACCTGCGATCCAAGGCGTGTGACATCTTG
GCAATTGATAAGTCACTCACGCCGGTTACTTTGGTGCTTGCCGAAGACGG
CACAATAGTAGACGACGACGATTACTTCCTCTGCCTTCCGTCAAACACGA
AGTTTGTTGCACTTGCGAGTAATGAGAAATGGGCTTACAACAATTCAGAT
GGTGGCACCGCCTGGATTAGCCAGGAATCTTTCGACGTAGATGAAACAGA
CTCTGGGGCCGGTCTCAAATGGAAGAACGTTGCCCGGCAGCTTAAAGAAG
ATCTGTCTAGCATAATCCTGCTCTCTGAGGAGGATCTGCAGATGCTGGTGG
ATGCGCCCTGTAGTGATTTGGCCCAAGAACTTCGACAAAGTTGTGCAACT
GTACAACGGCTCCAACACACATTGCAGCAGGTGCTGGACCAGCGAGAGG
AGGTGAGGCAGAGCAAGCAACTTCTTCAACTCTACCTCCAAGCTCTTGAG
AAAGAGGGCTCATTGCTCTCAAAACAAGAAGAGTCTAAAGCAGCGTTTGG
TGAGGAAGTCGACGCTGTAGACACTGGAATTTCAAGAGAGACTAGTTCCG
ATGTTGCGCTTGCTAGTCATATTCTTACAGCTCTGAGAGAGAAGCAAGCTC
CAGAACTCTCTCTCAGCTCCCAAGACCTCGAACTTGTTACTAAAGAAGAC
CCGAAGGCGCTGGCAGTCGCTCTTAACTGGGACATCAAGAAGACAGAGA
CaGTTCAAGAGGCATGTGAACGGGAGCTGGCATTGCGCCTCCAACAAACT
CAATCCCTGCATTCCCTGCGGTCCATAAGCGCCTCAAAAGCAAGCCCTCC
AGGGGATCTGCAGAATCCTAAGAGGGCGCGGCAAGATCCCACGggcagtggct
cgggctcggggtccggcggaTTCAATGTCTTAATGGTTCATAAGCGAAGCCATACTG
GTGAACGCCCATTGCAGTGCGAAATATGCGGCTTTACCTGCCGCCAGAAA
GGTAACCTCCTCCGCCACATTAAACTGCACACAGGGGAAAAACCTTTTAA
GTGTCACCTCTGCAACTATGCATGCCAAAGAAGAGATGCGCTCTCCGGAG
GCGGCGGAGAGGGCAGAGGAAGTCTTCTAACATGCGGTGACGTGGAGGA
GAATCCCGGCCCTAGGATGCTCCAGAAGCCCAAGAGCGTGAAGCTGgccGC
CCTGCGCAGCCCGAGGAAGTTCGGCGTGGCTGGCCGGAGCTGCCAGGAG
GTGCTGCGCAAGGGCTGTCTCCGCTTCCAGCTCCCTGAGCGCGGTTCCCGG
CTGTGCCTGtacGAGGATGGCACGGAGCTGACGGAAGATTACTTCCCCAGT
GTTCCCGACAACGCCGAGCTGGTGCTGCTCACCTTGGGCCAGGCCTGGCA
GGGCTATGTGAGCGACATCAGGCGCTTCCTCAGTGCATTTCACGAGCCAC
AGGTGGGGCTCATCCAGGCCGCCCAGCAGCTGCTGTGTGATGAGCAGGCC
CCACAGAGGCAGAGGCTGCTGGCTGACCTCCTGCACAACGTCAGCCAGAA
CATCGCGGCCGAGACCCGGGCTGAGGACCCGCCGTGGTTTGAAGGCTTGG
AGTCCCGATTTCAGAGCAAGTCTGGCTATCTGAGATACAGCTGTGAGAGC
CGGATCCGGAGTTACCTGAGGGAGGTGAGCTCCTACCCCTCCACGGTGGG
TGCGGAGGCTCAGGAGGAATTCCTGCGGGTCCTCGGCTCCATGTGCCAGA
GGCTCCGGTCCATGCAGTACAATGGCAGCTACTTCGACAGAGGAGCCAAG
GGCGGCAGCCGCCTCTGCACACCGGAAGGCTGGTTCTCCTGCCAGGGTCC
CTTTGACATGGACAGCTGCTTATCAAGACACTCCATCAACCCCTACAGTA
ACAGGGAGAGCAGGATCCTCTTCAGCACCTGGAACCTGGATCACATAATA
GAAAAGAAACGCACCATCATTCCTACACTGGTGGAAGCAATTAAGGAAC
AAGATGGAAGAGAAGTGGACTGGGAGTATTTTTATGGCCTGCTTTTTACCT
CAGAGAACCTAAAACTAGTGCACATTGTCTGCCATAAGAAAACCACCCAC
AAGCTCAACTGTGACCCAAGCAGAATCTACAAACCCCAGACAAGGTTGAA
GCGGAAGCAGCCTGTGCGGAAACGCCAG
(SEQ ID NO: 28)
MEVTGDAGVPESGEIRTLKPCLLRRNYSREQHGVAASCLEDLRSKACDILAID
KSLTPVTLVLAEDGTIVDDDDYFLCLPSNTKFVALASNEKWAYNNSDGGTA
WISQESFDVDETDSGAGLKWKNVARQLKEDLSSIILLSEEDLQMLVDAPCSDL
AQELRQSCATVQRLQHTLQQVLDQREEVRQSKQLLQLYLQALEKEGSLLSK
QEESKAAFGEEVDAVDTGISRETSSDVALASHILTALREKQAPELSLSSQDLEL
VTKEDPKALAVALNWDIKKTETVQEACERELALRLQQTQSLHSLRSISASKAS
PPGDLQNPKRARQDPTGSGSGSGSGGFNVLMVHKRSHTGERPLQCEICGFTC
RQKGNLLRHIKLHTGEKPFKCHLCNYACQRRDALSGGGGEGRGSLLTCGDV
EENPGPRMLQKPKSVKLAALRSPRKFGVAGRSCQEVLRKGCLRFQLPERGSR
LCLYEDGTELTEDYFPSVPDNAELVLLTLGQAWQGYVSDIRRFLSAFHEPQV
GLIQAAQQLLCDEQAPQRQRLLADLLHNVSQNIAAETRAEDPPWFEGLESRF
QSKSGYLRYSCESRIRSYLREVSSYPSTVGAEAQEEFLRVLGSMCQRLRSMQY
NGSYFDRGAKGGSRLCTPEGWFSCQGPFDMDSCLSRHSINPYSNRESRILFST
WNLDHIIEKKRTIIPTLVEAIKEQDGREVDWEYFYGLLFTSENLKLVHIVCHKK
TTHKLNCDPSRIYKPQTRLKRKQPVRKRQ
ICAD-degron-CADp.R11W/E68A
(SEQ ID NO: 29)
ATGGAAGTGACTGGTGATGCCGGTGTCCCAGAGAGCGGCGAGATCAGGA
CTCTTAAACCATGCCTGCTTAGGCGAAACTACAGCCGAGAGCAACATGGT
GTTGCTGCTAGTTGCTTGGAAGACCTGCGATCCAAGGCGTGTGACATCTTG
GCAATTGATAAGTCACTCACGCCGGTTACTTTGGTGCTTGCCGAAGACGG
CACAATAGTAGACGACGACGATTACTTCCTCTGCCTTCCGTCAAACACGA
AGTTTGTTGCACTTGCGAGTAATGAGAAATGGGCTTACAACAATTCAGAT
GGTGGCACCGCCTGGATTAGCCAGGAATCTTTCGACGTAGATGAAACAGA
CTCTGGGGCCGGTCTCAAATGGAAGAACGTTGCCCGGCAGCTTAAAGAAG
ATCTGTCTAGCATAATCCTGCTCTCTGAGGAGGATCTGCAGATGCTGGTGG
ATGCGCCCTGTAGTGATTTGGCCCAAGAACTTCGACAAAGTTGTGCAACT
GTACAACGGCTCCAACACACATTGCAGCAGGTGCTGGACCAGCGAGAGG
AGGTGAGGCAGAGCAAGCAACTTCTTCAACTCTACCTCCAAGCTCTTGAG
AAAGAGGGCTCATTGCTCTCAAAACAAGAAGAGTCTAAAGCAGCGTTTGG
TGAGGAAGTCGACGCTGTAGACACTGGAATTTCAAGAGAGACTAGTTCCG
ATGTTGCGCTTGCTAGTCATATTCTTACAGCTCTGAGAGAGAAGCAAGCTC
CAGAACTCTCTCTCAGCTCCCAAGACCTCGAACTTGTTACTAAAGAAGAC
CCGAAGGCGCTGGCAGTCGCTCTTAACTGGGACATCAAGAAGACAGAGA
CaGTTCAAGAGGCATGTGAACGGGAGCTGGCATTGCGCCTCCAACAAACT
CAATCCCTGCATTCCCTGCGGTCCATAAGCGCCTCAAAAGCAAGCCCTCC
AGGGGATCTGCAGAATCCTAAGAGGGCGCGGCAAGATCCCACGggcagtggct
cgggctcggggtccggcggaTTCAATGTCTTAATGGTTCATAAGCGAAGCCATACTG
GTGAACGCCCATTGCAGTGCGAAATATGCGGCTTTACCTGCCGCCAGAAA
GGTAACCTCCTCCGCCACATTAAACTGCACACAGGGGAAAAACCTTTTAA
GTGTCACCTCTGCAACTATGCATGCCAAAGAAGAGATGCGCTCTCCGGAG
GCGGCGGAGAGGGCAGAGGAAGTCTTCTAACATGCGGTGACGTGGAGGA
GAATCCCGGCCCTAGGATGCTCCAGAAGCCCAAGAGCGTGAAGCTGGGG
CCCTGCGCAGCCCGAGGAAGTTCGGCGTGGCTGGCCGGAGCTGCCAGGAG
GTGCTGCGCAAGGGCTGTCTCCGCTTCCAGCTCCCTGAGCGCGGTTCCCGG
CTGTGCCTGTACGAGGATGGCACGGAGCTGACGGAAGATTACTTCCCCAG
TGTTCCCGACAACGCCGccCTGGTGCTGCTCACCTTGGGCCAGGCCTGGCA
GGGCTATGTGAGCGACATCAGGCGCTTCCTCAGTGCATTTCACGAGCCAC
AGGTGGGGCTCATCCAGGCCGCCCAGCAGCTGCTGTGTGATGAGCAGGCC
CCACAGAGGCAGAGGCTGCTGGCTGACCTCCTGCACAACGTCAGCCAGAA
CATCGCGGCCGAGAGCTATGTGAGCGACATCAGGCGCTTCCTCAGTGCAT
TTCACGAGCCACAGGTGGGGCTCATCCAGGCCGCCCAGCAGCTGCTGTGT
GATGAGCAGGCCCCACAGAGGCAGAGGCTGCTGGCTGACCTCCTGCACAA
CGTCAGCCAGAACATCGCGGCCGAGACCCGGGCTGAGGACCCGCCGTGGT
TTGAAGGCTTGGAGTCCCGATTTCAGAGCAAGTCTGGCTATCTGAGATAC
AGCTGTGAGAGCCGGATCCGGAGTTACCTGAGGGAGGTGAGCTCCTACCC
CTCCACGGTGGGTGCGGAGGCTCAGGAGGAATTCCTGCGGGTCCTCGGCT
CCATGTGCCAGAGGCTCCGGTCCATGCAGTACAATGGCAGCTACTTCGAC
AGAGGAGCCAAGGGCGGCAGCCGCCTCTGCACACCGGAAGGCTGGTTCTC
CTGCCAGGGTCCCTTTGACATGGACAGCTGCTTATCAAGACACTCCATCA
ACCCCTACAGTAACAGGGAGAGCAGGATCCTCTTCAGCACCTGGAACCTG
GATCACATAATAGAAAAGAAACGCACCATCATTCCTACACTGGTGGAAGC
AATTAAGGAACAAGATGGAAGAGAAGTGGACTGGGAGTATTTTTATGGCC
TGCTTTTTACCTCAGAGAACCTAAAACTAGTGCACATTGTCTGCCATAAGA
AAACCACCCACAAGCTCAACTGTGACCCAAGCAGAATCTACAAACCCCAG
ACAAGGTTGAAGCGGAAGCAGCCTGTGCGGAAACGCCAG
(SEQ ID NO: 30)
MEVTGDAGVPESGEIRTLKPCLLRRNYSREQHGVAASCLEDLRSKACDILAID
KSLTPVTLVLAEDGTIVDDDDYFLCLPSNTKFVALASNEKWAYNNSDGGTA
WISQESFDVDETDSGAGLKWKNVARQLKEDLSSIILLSEEDLQMLVDAPCSDL
AQELRQSCATVQRLQHTLQQVLDQREEVRQSKQLLQLYLQALEKEGSLLSK
QEESKAAFGEEVDAVDTGISRETSSDVALASHILTALREKQAPELSLSSQDLEL
VTKEDPKALAVALNWDIKKTETVQEACERELALRLQQTQSLHSLRSISASKAS
PPGDLQNPKRARQDPTGSGSGSGSGGFNVLMVHKRSHTGERPLQCEICGFTC
RQKGNLLRHIKLHTGEKPFKCHLCNYACQRRDALSGGGGEGRGSLLTCGDV
EENPGPRMLQKPKSVKLWALRSPRKFGVAGRSCQEVLRKGCLRFQLPERGSR
LCLYEDGTELTEDYFPSVPDNAALVLLTLGQAWQGYVSDIRRFLSAFHEPQV
GLIQAAQQLLCDEQAPQRQRLLADLLHNVSQNIAAESYVSDIRRFLSAFHEPQ
VGLIQAAQQLLCDEQAPQRQRLLADLLHNVSQNIAAETRAEDPPWFEGLESR
FQSKSGYLRYSCESRIRSYLREVSSYPSTVGAEAQEEFLRVLGSMCQRLRSMQ
YNGSYFDRGAKGGSRLCTPEGWFSCQGPFDMDSCLSRHSINPYSNRESRILFS
TWNLDHIIEKKRTIIPTLVEAIKEQDGREVDWEYFYGLLFTSENLKLVHIVCHK
KTTHKLNCDPSRIYKPQTRLKRKQPVRKRQ
Degron aka ″superdegron″ aka ZFP91-IKZF3 degron
(SEQ ID NO: 31)
TTCAATGTCTTAATGGTTCATAAGCGAAGCCATACTGGTGAACGCCCATTG
CAGTGCGAAATATGCGGCTTTACCTGCCGCCAGAAAGGTAACCTCCTCCG
CCACATTAAACTGCACACAGGGGAAAAACCTTTTAAGTGTCACCTCTGCA
ACTATGCATGCCAAAGAAGAGATGCGCTC
(SEQ ID NO: 32)
FNVLMVHKRSHTGERPLQCEICGFTCRQKGNLLRHIKLHTGEKPFKCHLCNY
ACQRRDAL
913iK0
(SEQ ID NO: 14)
TTCAATGTCTTAATGGTTCATcggCGAAGCCATACTGGTGAACGCCCATTGC
AGTGCGAAATATGCGGCTTTACCTGCCGCCAGcgcGGTAACCTCCTCCGCC
ACATTcgtCTGCACACAGGGGAAcggCCTTTTcggTGTCACCTCTGCAACTATG
CATGCCAAAGAAGAGATGCGCTC
(SEQ ID NO: 33)
FNVLMVHRRSHTGERPLQCEICGFTCRQRGNLLRHIRLHTGERPFRCHLCNYA
CQRRDAL
EF1alpha promoter
(SEQ ID NO: 34)
GGCTCCGGTGCCCGTCAGTGGGCAGAGCGCACATCGCCCACAGTCC
CCGAGAAGTTGGGGGGAGGGGTCGGCAATTGAACCGGTGCCTAGAGAAG
GTGGCGCGGGGTAAACTGGGAAAGTGATGTCGTGTACTGGCTCCGCCTTT
TTCCCGAGGGTGGGGGAGAACCGTATATAAGTGCAGTAGTCGCCGTGAAC
GTTCTTTTTCGCAACGGGTTTGCCGCCAGAACACAGGTAAGTGCCGTGTGT
GGTTCCCGCGGGCCTGGCCTCTTTACGGGTTATGGCCCTTGCGTGCCTTGA
ATTACTTCCACCTGGCTGCAGTACGTGATTCTTGATCCCGAGCTTCGGGTT
GGAAGTGGGTGGGAGAGTTCGAGGCCTTGCGCTTAAGGAGCCCCTTCGCC
TCGTGCTTGAGTTGAGGCCTGGCCTGGGCGCTGGGGCCGCCGCGTGCGAA
TCTGGTGGCACCTTCGCGCCTGTCTCGCTGCTTTCGATAAGTCTCTAGCCA
TTTAAAATTTTTGATGACCTGCTGCGACGCTTTTTTTCTGGCAAGATAGTCT
TGTAAATGCGGGCCAAGATCTGCACACTGGTATTTCGGTTTTTGGGGCCGC
GGGCGGCGACGGGGCCCGTGCGTCCCAGCGCACATGTTCGGCGAGGCGG
GGCCTGCGAGCGCGGCCACCGAGAATCGGACGGGGGTAGTCTCAAGCTG
GCCGGCCTGCTCTGGTGCCTGGCCTCGCGCCGCCGTGTATCGCCCCGCCCT
GGGCGGCAAGGCTGGCCCGGTCGGCACCAGTTGCGTGAGCGGAAAGATG
GCCGCTTCCCGGCCCTGCTGCAGGGAGCTCAAAATGGAGGACGCGGCGCT
CGGGAGAGCGGGCGGGTGAGTCACCCACACAAAGGAAAAGGGCCTTTCC
GTCCTCAGCCGTCGCTTCATGTGACTCCACGGAGTACCGGGCGCCGTCCA
GGCACCTCGATTAGTTCTCGAGCTTTTGGAGTACGTCGTCTTTAGGTTGGG
GGGAGGGGTTTTATGCGATGGAGTTTCCCCACACTGAGTGGGTGGAGACT
GAAGTTAGGCCAGCTTGGCACTTGATGTAATTCTCCTTGGAATTTGCCCTT
TTTGAGTTTGGATCTTGGTTCATTCTCAAGCCTCAGACAGTGGTTCAAAGT
TTTTTTCTTCCATTTCAGGTGTCGTGA
PGK promoter
(SEQ ID NO: 35)
TTGCGCCTTTTCCAAGGCAGCCCTGGGTTTGCGCAGGGACGCGGCTGCTCT
GGGCGTGGTTCCGGGAAACGCAGCGGCGCCGACCCTGGGTCTCGCACATT
CTTCACGTCCGTTCGCAGCGTCACCCGGATCTTCGCCGCTACCCTTGTGGG
CCCCCCGGCGACGCTTCCTGCTCCGCCCCTAAGTCGGGAAGGTTCCTTGCG
GTTCGCGGCGTGCCGGACGTGACAAACGGAAGCCGCACGACTCACTAGTA
CCCTCGCAGACGGACAGCGCCAGGGAGCAATGGCAGCGCGCCGACCGCG
ATGGGCTGTGGCCAATAGCGGCTGCTCAGCAGGGCGCGCCGAGAGCAGC
GGCCGGGAAGGGGCGGTGCGGGAGGCGGGGTGTGGGGCGGTAGTGTGGG
CCCTGTTCCTGCCCGCGCGGTGTTCCGCATTCTGCAAGCCTCCGGAGCGCA
CGTCGGCAGTCGGCTCCCTCGTTGACCGAATCACCGACCTCTCTCCCCAGG
G

### EXAMPLES

The invention is further described in the following examples, which do not limit the scope of the invention described in the claims.

### Materials and Methods

The following materials and methods were used in the Examples below.

### Vector Cloning

Transgenes were synthesized as linear DNA fragments and cloned into lentiviral vectors. Suicide switch candidates were cloned into a lentivector with an EF1alpha promoter cloning site, and C-terminal P2A and mTagBFP2 elements.

### Lentiviral Production

HEK 293 cells were cultured in RPMI 1640 medium with GLUTAMAX (L-alanyl-L-glutamine supplement) and HEPES supplemented with 10% fetal bovine serum (FBS), penicillin, and streptomycin. Lentiviral particles were produced in HEK 293 cells transduced with a mixture of, for each mL of HEK 293 cell culture media, 34 μL OPTIMEM (reduced-serum medium), 0.27 μg transfer plasmid, 0.45 μg VSV-G, 0.18 μg pSPAX2, and 3 μL of FUGENE HD (non-liposomal transfection reagent). For cell culture experiments, lentiviral particle-containing supernatant was collected 48 hours after transfection, passed through a 0.45 μM polyvinylidene difluoride (PVDF) filter, and applied to target cells or stored at −80 C. For primary human T cell experiments, lentiviral particle-containing supernatant was concentrated by ultracentrifugation at 25,000 RPM in a SW28 rotor, resuspended in approximately 1/100 of the starting volume in RPMI or PBS, and cryopreserved at −80 C.

### ICAD Degradation

An ICAD-degron sequence was cloned into a vector composed of a PGK promoter, followed by GFP, a glycine-serine linker, cloning sites, IRES, and mCherry. Jurkat T cells were transduced to express GFP-ICAD-degron and mCherry. Flow cytometry was used to assess mean GFP fluorescence intensity with and without lenalidomide treatment, using mCherry to gate on transduced cells.

### DNA Damage Assay

Cells were treated with the indicated lenalidomide concentrations and analyzed for gamma-H2AX positivity by intracellular flow according to manufacturer's specifications (BD Cytofix/Cytoperm).

### Competitive Proliferation Assay

Cells were stimulated weekly with an equal number of irradiated K562 cells transduced to express CD3 and CD28 scFvs. Flow cytometry was used to enumerate the percentage of mTagBFP2+ cells on days 0, 7, and 14.

### Cell Death Assays

Unless otherwise indicated, cell death assays with Jurkat or primary human T cells were performed by 48 hour incubation with the relevant drug and flow cytometry analysis for the marker of transduction.

### Live Cell Imaging Assay (INCUCYTE)

On day −1, tissue culture plates were coated with anti-CD71 antibody diluted in PBS. On day 0, the plates were washed with PBS and then Nalm6-luciferase-GFP cells were added to the coated plates. Four hours later, primary human CAR T cells were added at a 1:1 ratio with the Nalm6 cells. Plates were imaged every hour for at least 90 hours (INCUCYTE ZOOM, live-cell analysis system). Total green, red, and blue object areas were collected, indicative of GFP+ tumor and mCherry+/mTagBFP2+ CAR and/or suicide switch T cell area.

### Example 1. Lenalidomide Suicide Switches

We envisioned a suicide switch in which a pair of transgenes would be expressed, 1) a pro-apoptotic gene and 2) a lenalidomide-responsive degron-tagged anti-apoptotic gene. These would constitute a stoichiometric pair that would be inert until lenalidomide is added, resulting in anti-apoptotic protein degradation, an excess of pro-apoptotic proteins, activated apoptotic signaling, and cell death.

Pairs of pro/anti-apoptotic proteins were evaluated for their ability to work as an effective suicide switch. BCL-xL and BIM transgene pairs were toxic to Jurkat cells transduced to express these lentiviruses, as assessed by total cell number 48 hours after high-titer transduction, see FIG. 1A. Lenalidomide did not alter the growth and survival of cells transduced to express pairs of BCL-xL and variants of of PUMA fused to an E3 recruitment domain 913iK0, see FIG. 1B. In this design, the goal was for the pro-apoptotic protein PUMA to bind endogenous anti-apoptotic proteins, and for drug-dependent recruitment to the E3 to result in anti-apoptotic protein ubiquitination, degradation, and cell death. This did not work.

To determine whether a specific position of the degron is required for efficient lentiviral transduction, a number of vectors were tested with varying positions for the degron, as shown in Table 1.

TABLE 1
Vectors tested, varying position of degron

| Construct # | Promoter | Transgenes | Marker | Label (nt) |
|---|---|---|---|---|
| #071 | pEF1a | ICAD-degron-CAD codon opt. | T2A-mTagBFP2 | 10055 |
| #073 | pEF1a | ICAD-degron-CAD p.R11W | T2A-mTagBFP2 | 10055 |
| #074 | pEF1a | degron-ICAD-CAD p.R11W | T2A-mTagBFP2 | 10055 |
| #075 | pEF1a | ICAD-degron-CAD | T2A-mTagBFP2 | 10055 |
| #076 | pEF1a | degron-ICAD-CAD | T2A-mTagBFP2 | 10055 |

As shown in FIG. 2, transduction efficiency of Jurkat cells with the indicated lentiviral supernatant varied, indicating that cells could not tolerate the ICAD-CAD transgene pair when the degron was on the N terminus of ICAD. These results indicated that C-terminal positioning of the degron was required for efficient lentiviral transduction, likely because the degron-ICAD conformation is unstable, resulting in toxicity both to the viral producer cells and any transduced Jurkat cells (FIG. 2).

To test whether variations in CAD modify the performance of CAD/ICAD suicide switch, a primary human T cell four-day growth assay was performed comparing vector control, and then three modifications of ICAD-degron 2A CAD expression, differentiated by the CAD sequences. As shown in FIG. 3, the p.R11W mutation enhanced drug-independent cell fitness. The ICAD-degron 2A CAD p.R11W is referred to as the lenalidomide “kill switch” or “suicide switch” in the present figures.

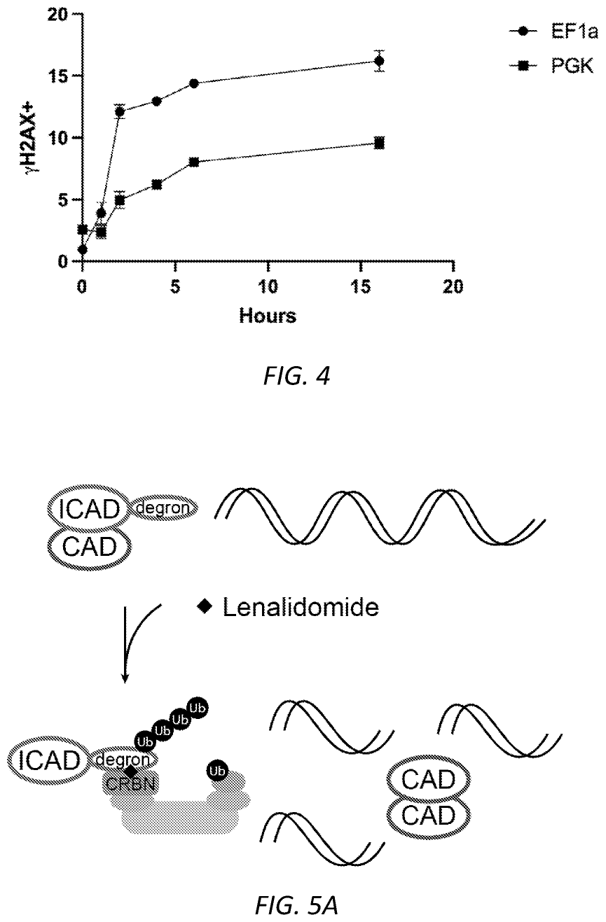

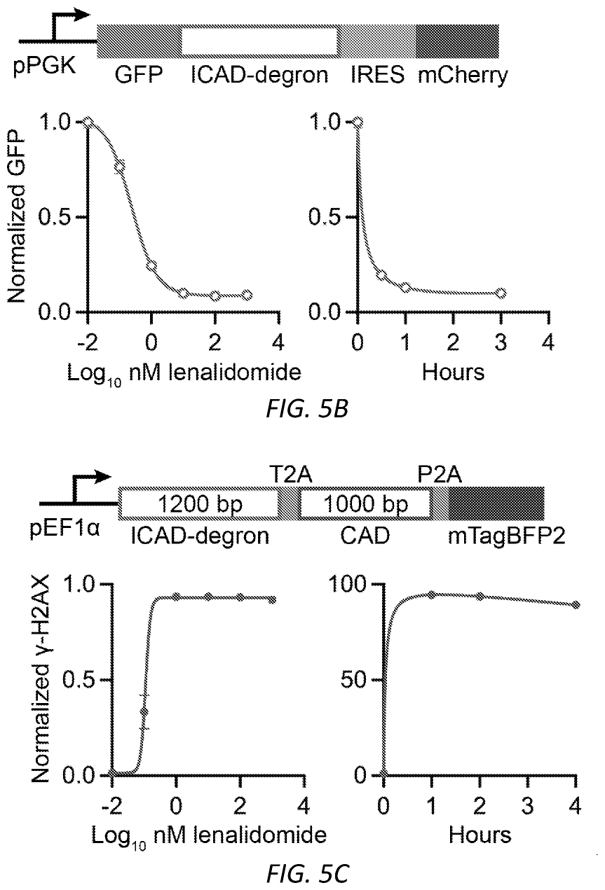

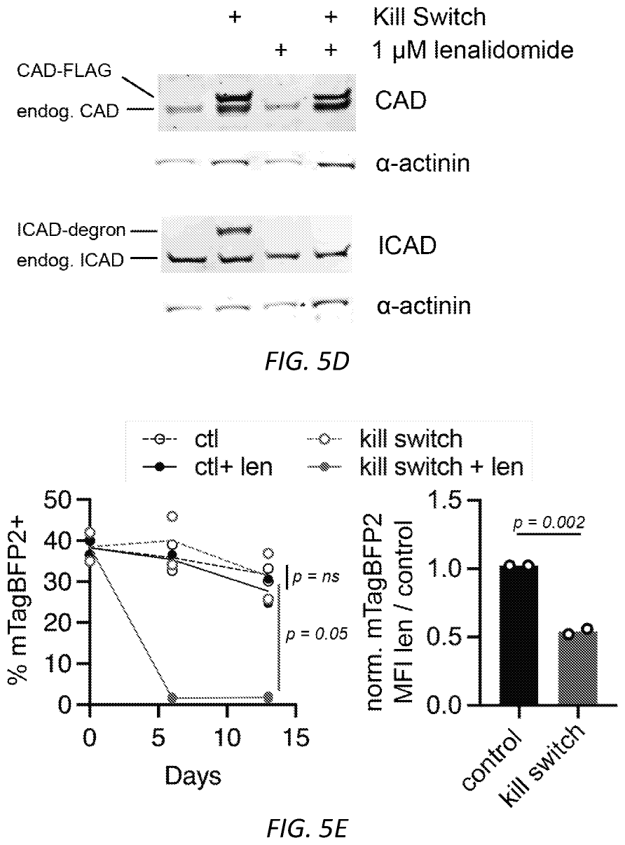

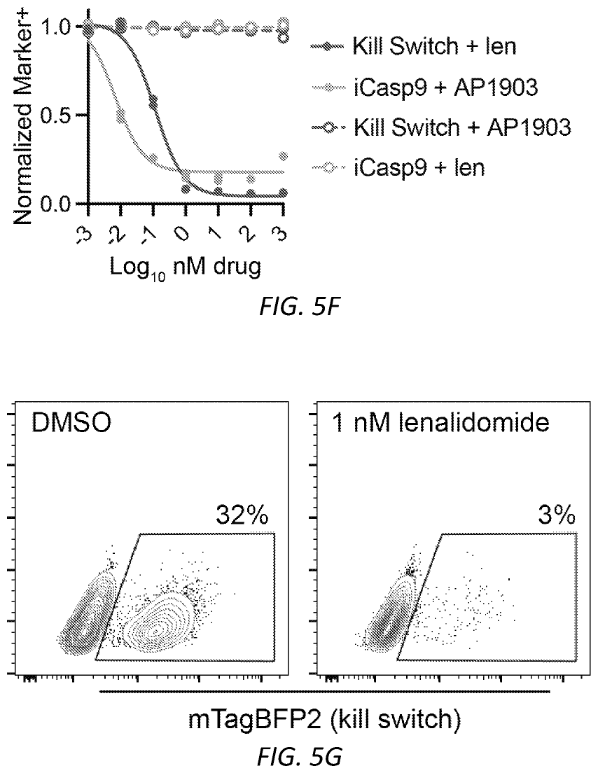

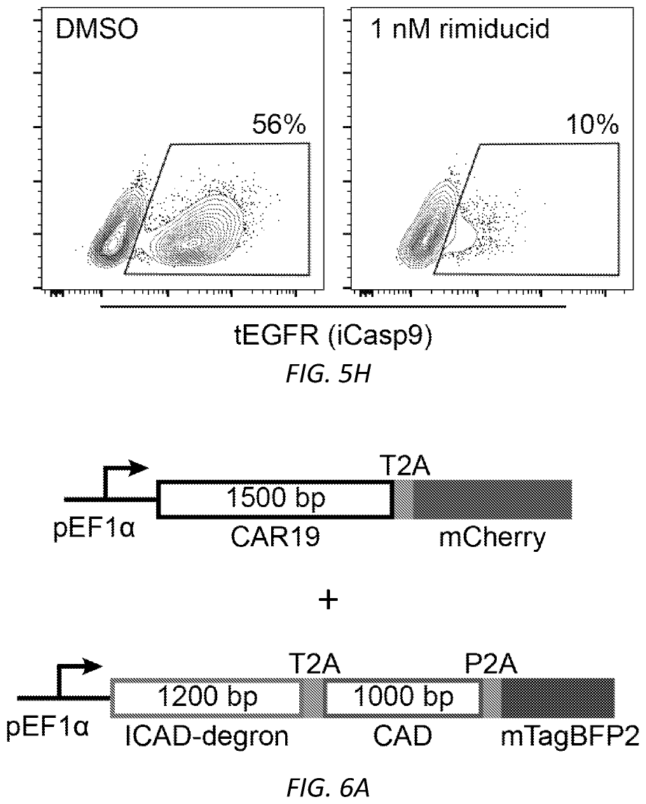

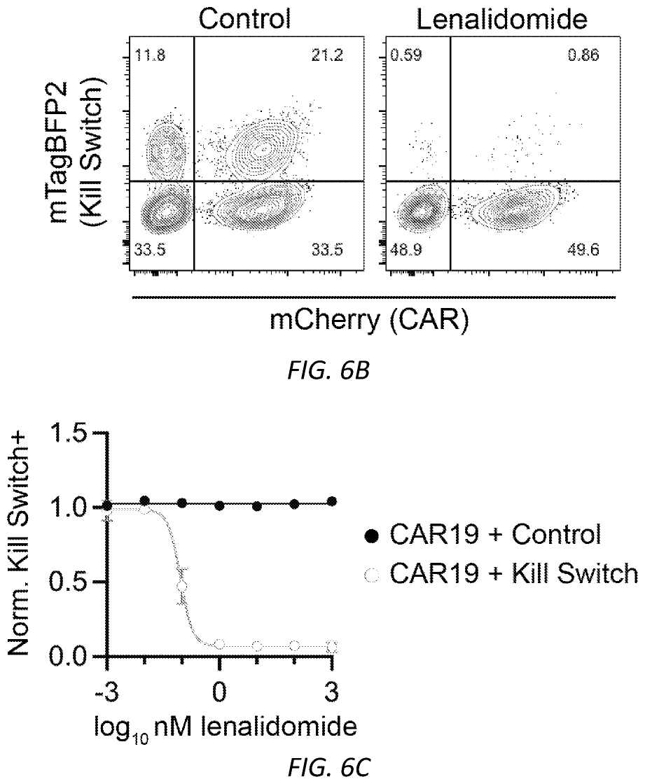

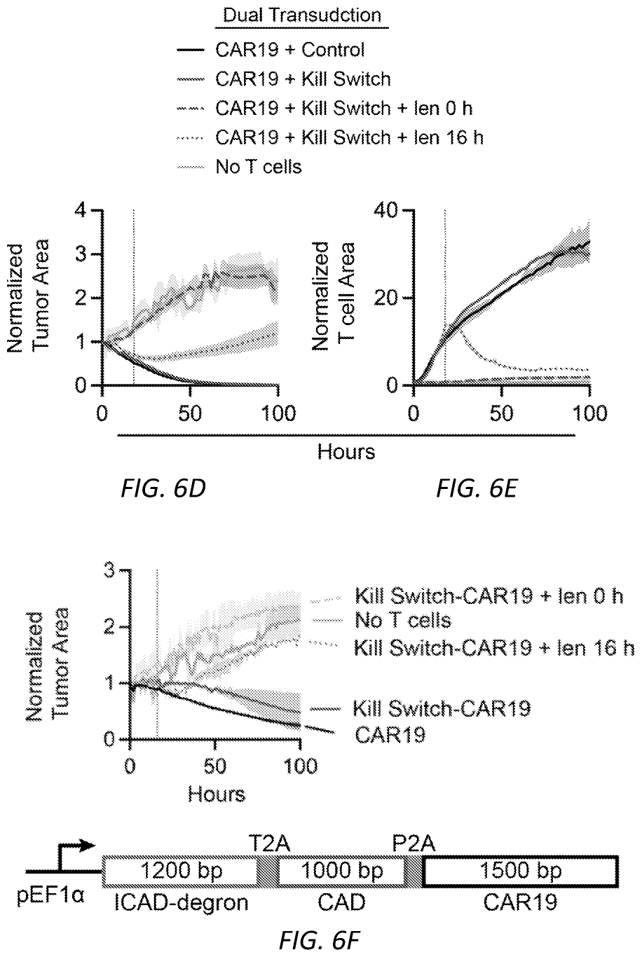

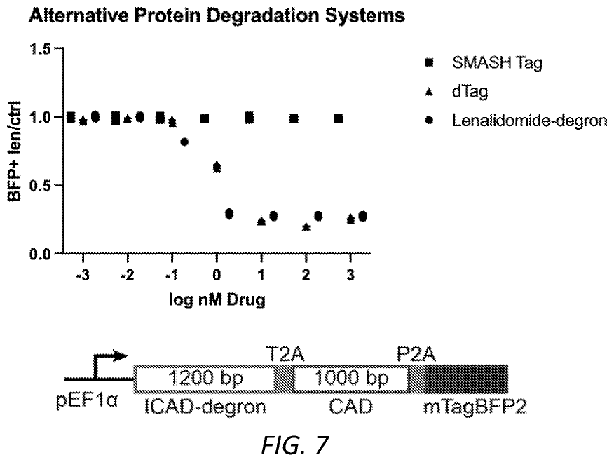

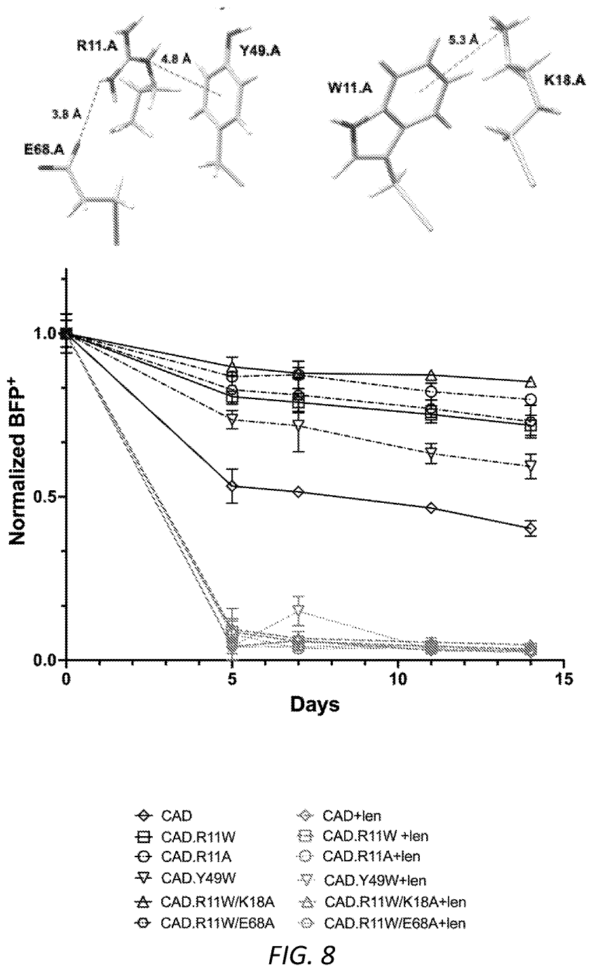

To determine whether promoter strength modifies ICADdegron-CAD suicide switch efficiency, Jurkat cells were transduced to express the ICAD-degron 2A CAD p.R11W suicide switch was expressed, driven by an EF1alpha or PGK promoter. As shown in FIG. 4, an enhanced DNA damage response was obtained from the version with the stronger EF1alpha promoter.

The ability of an ICAD-degron CAD transgene pair to act as a lenalidomide-inducible suicide switch was evaluated. Without wishing to be bound by theory, FIG. 5A shows a schema of the believed mechanism of action, with the ICAD-degron—CAD suicide switch shown at top left, and the intact genomic DNA at top right. The addition of lenalidomide (black diamonds) causes CRBN-dependent ubiquitination of the degron (bottom left) and subsequent disassociation of CAD and ICAD, allowing active CAD to degrade genomic DNA and promote apoptosis of the cell (bottom right).

To validate lenalidomide-dependent ICAD-degron depletion, we created a reporter system in which mCherry+ transduced cells could be identified and quantified for GFP-ICAD-degron fusion protein fluorescence (construct illustrated in FIG. 5B, top). Assessment of GFP-ICAD-degron reporter fluorescence was performed in Jurkat cells. As shown in FIG. 5B, exposure to a range of drug concentrations overnight produced a dose-dependent response. A timecourse experiment was performed after 1000 nM lenalidomide addition (FIG. 5B). ICAD-degron protein degradation occurred with sub-nanomolar drug concentrations and largely within one hour.

To assess the impact of the lenalidomide suicide switch on DNA damage sensing, Jurkat cells expressing the switch were treated with lenalidomide and analyzed for the accumulation of gamma-H2Ax, a marker of DNA damage. A schema for a lenalidomide suicide switch is shown at the top of FIG. 5C. Gamma H2AX fluorescence was used as a marker of DNA damage assessed across a range of drug concentrations administered overnight (FIG. 5C, bottom left) or assessed as a timecourse after 1000 nM lenalidomide addition (FIG. 5C, bottom right). DNA damage sensing was activated with 0.1 nM lenalidomide, fully activated at 1 nM, and occurred within one hour (FIG. 5C).

To assess the dynamics of endogenous and transgenic proteins in the switch, an ICAD-degron-T2A-CADp.R11W-FLAG system was generated. Western blotting was performed to assess the abundance of endogenous and transgenic overexpressed proteins with and without 4 hours of exposure to 1000 nM lenalidomide addition. Lenalidomide specifically destabilized the ICAD-degron fusion protein (FIG. 5D), creating an excess of CAD.

To assess the long-term fitness of lenalidomide suicide switch-transduced primary human CAR T cells or cells transduced with a mTagBFP2+ control lentivector, these cells were transduced, expanded for 2 weeks, and then repetitively stimulated with dynabeads. Control and lenalidomide suicide switch T cell proliferation (in competition with untransduced cells) was comparable without lenalidomide and significantly depleted with lenalidomide. The surviving mTagBFP2+ suicide switch cells had reduced fluorescent protein intensity, indicating that lower transgene expression is associated with survival in the minor subpopulation of surviving cells (FIG. 5E).

To compare the primary T cell clearance with the lenalidomide suicide switch or iCaspase9, a head-to-head comparison was performed with a concentration range of each controller drug, lenalidomide and AP1903. Cell depletion (normalized to untreated cells) of primary human T cells expressing the CAD/ICAD versus iCasp9 suicide switches was evaluated across dose ranges of AP1903 and lenalidomide. The results, shown in FIGS. 5F-H, demonstrated more complete cell killing with the CAD/ICAD switch.

Control of CAR T cell function with a lenalidomide suicide switch was also evaluated. FIG. 6A shows a schema of the dual-vector CAR and ICAD-degron/CAD suicide switch constructs used in this experiment, with a CAR construct that included CAR 19 and mCherry separated by a T2A element and driven by an EF1α promoter, and a ICAD-degron T2A CAD R11W P2A mTagBFP2 construct, also driven by an EF1α promoter.

FIG. 6B shows successful depletion of ICAD-degron/CAD suicide switch-positive primary human T cells after 1 uM lenalidomide treatment, and depletion of sorted CAR+ ICAD-degron/CAD suicide switch+primary human T cells across a dose range of lenalidomide is shown in FIG. 6C.

Dual-transduced lenalidomide suicide switch CAR T cells or control mTagBFP2+ CAR T cells were purified by FACS. Tumor cell cytolysis was evaluated by Incucyte live cell imaging co-culture assay with NALM6 tumor cells. Anti-tumor potency was comparable with control or suicide switch CAR T cells. Immediate lenalidomide prevented tumor cell cytolysis. 16 hour delayed lenalidomide ablated tumor cell depletion, with subsequent NALM6 proliferation (FIG. 6D). These data indicate that the lenalidomide suicide switch can be used to rapidly ablate the function of CAR T cells.

In the same assay, CAR T cell proliferation was assessed, and was comparable without drug between control and suicide switch CAR T cells. Immediate lenalidomide prevented suicide switch CAR T cell expansion, and 16 hour delayed lenalidomide resulted in depletion of the CAR T cells within the next 24 hours without rebound. In summary, the suicide switch can be used to deplete CAR T cells (FIG. 6E).

We also generated a tri-cistronic suicide switch CAR lentivector. The same Incucyte live cell imaging co-culture assay with NALM6 tumor cells and single lentivector ICAD-degron/CAD/CAR was performed in primary human T cells. Anti-tumor cytolysis was comparable to a conventional CAR without lenalidomide. With lenalidomide, anti-tumor cytolysis was prevented. These data indicate that the suicide switch can be co-delivered with a therapeutic (and potentially toxic) gene in a single lentivector in a gene- and cell-based therapy (FIG. 6F).

To determine whether the ICAD-degron CAD suicide switch design is generalizable to multiple degron systems, three different degron tags (SMASH, dTag, superdegron) were fused to ICAD to facilitate ICAD degradation. The “lenalidomide degron” is a ZFP91-IKZF3 zinc finger-based degron described in WO2019089592. Jurkat cells were transduced to express each lentivector, and then tested for loss of the BFP+ cells after 48 hours of exposure to controller drugs (ganciclovir for the SMASH tag, dTAG-13 for the dTAG, and lenalidomide). The results, shown in FIG. 7, demonstrated that the IKZF3 and dTag, but not the SMASH tag, allowed for drug-dependent cell depletion. These data demonstrate 1) that not all degrons work in this system, and 2) ICAD-degron-CAD does work with other degron domains.

To assess whether further modification of CAD could further enhance the suicide switch, we performed structural modeling of CAD, identifying side chain interactions with R11 and W 11 in the mutant form. A competitive growth assay was performed using modified lenalidomide suicide switches (as shown in FIG. 7 comprising CAD R11W) in Jurkat cells, with 1 uM lenalidomide (dashed lines) or without (solid lines), normalized to % transduced on day 0. The results, shown in FIG. 8, showed that several mutations, including mutations at R11 (e.g., R11W and R11A), Y49 (e.g., Y49W), and combinations of R11/K18 (e.g., R11W/K18A) or R11/E68 (e.g., R11W/E68A) improved cell viability in the absence of lenalidomide, as indicated by normalized fluorescence. The compound mutation of ICAD-degron-T2A-CADp.R11W;K18A demonstrated the most improved drug-independent competitive growth and efficient cell depletion.

### REFERENCES

1. M. V. Maus, S. Alexander, M. R. Bishop, J. N. Brudno, C. Callahan, M. L. Davila, C. Diamonte, J. Dietrich, J. C. Fitzgerald, M. J. Frigault, T. J. Fry, J. L. Holter-Chakrabarty, K. V. Komanduri, D. W. Lee, F. L. Locke, S. L. Maude, P. L. McCarthy, E. Mead, S. S. Neelapu, T. G. Neilan, B. D. Santomasso, E. J. Shpall, D. T. Teachey, C. J. Turtle, T. Whitehead, S. A. Grupp, Society for Immunotherapy of Cancer (SITC) clinical practice guideline on immune effector cell-related adverse events. J Immunother Cancer 8, e001511 (2020).
2. O. V. Oekelen, A. Aleman, B. Upadhyaya, S. Schnakenberg, D. Madduri, S. Gavane, J. Teruya-Feldstein, J. F. Crary, M. E. Fowkes, C. B. Stacy, S. Kim-Schulze, A. Rahman, A. Lagana, J. D. Brody, M. Merad, S. Jagannath, S. Parekh, Neurocognitive and hypokinetic movement disorder with features of parkinsonism after BCMA-targeting CAR-T cell therapy. Nat Med 27, 2099-2103 (2021).
3. R. G. Majzner, S. Ramakrishna, K. W. Yeom, S. Patel, H. Chinnasamy, L. M. Schultz, R. M. Richards, L. Jiang, V. Barsan, R. Mancusi, A. C. Geraghty, Z. Good, A. Y. Mochizuki, S. M. Gillespie, A. M. S. Toland, J. Mahdi, A. Reschke, E. Nie, I. J. Chau, M. C. Rotiroti, C. W. Mount, C. Baggott, S. Mavroukakis, E. Egeler, J. Moon, C. Erickson, S. Green, M. Kunicki, M. Fujimoto, Z. Ehlinger, W. Reynolds, S. Kurra, K. E. Warren, S. Prabhu, H. Vogel, L. Rasmussen, T. T. Cornell, S. Partap, P. G. Fisher, C. J. Campen, M. G. Filbin, G. Grant, B. Sahaf, K. L. Davis, S. A. Feldman, C. L. Mackall, M. Monje, GD2-CAR T cell therapy for H3K27M-mutated diffuse midline gliomas. Nature, 1-10 (2022).
4. D. Mougiakakos, G. Kronke, S. Volkl, S. Kretschmann, M. Aigner, S. Kharboutli, S. Boltz, B. Manger, A. Mackensen, G. Schett, CD19-Targeted CAR T Cells in Refractory Systemic Lupus Erythematosus. New Engl J Med 385, 567-569 (2021).
5. A. C. Sahillioglu, T. N. Schumacher, Safety switches for adoptive cell therapy. Curr Opin Immunol (2021), doi:10.1016/j.coi.2021.07.002.
6. M. Jan, A. S. Sperling, B. L. Ebert, Cancer therapies based on targeted protein degradation lessons learned with lenalidomide. Nat Rev Clin Oncol, 1-17 (2021).
7. J. Kronke, N. D. Udeshi, A. Narla, P. Grauman, S. N. Hurst, M. McConkey, T. Svinkina, D. Heckl, E. Comer, X. Li, C. Ciarlo, E. Hartman, N. Munshi, M. Schenone, S. L. Schreiber, S. A. Carr, B. L. Ebert, Lenalidomide causes selective degradation of IKZF1 and IKZF3 in multiple myeloma cells. Sci New York N Y 343, 301-5 (2013).
8. G. Lu, R. E. Middleton, H. Sun, M. Naniong, C. J. Ott, C. S. Mitsiades, K.-K. Wong, J. E. Bradner, W. G. Kaelin, The myeloma drug lenalidomide promotes the cereblon-dependent destruction of Ikaros proteins. Sci New York N Y 343, 305-9 (2013).
9. M. Jan, I. Scarfo, R. C. Larson, A. Walker, A. Schmidts, A. A. Guirguis, J. A. Gasser, M. Slabicki, A. A. Bouffard, A. P. Castano, M. C. Kann, M. L. Cabral, A. Tepper, D. E. Grinshpun, A. S. Sperling, T. Kyung, Q. L. Sievers, M. E. Birnbaum, M. V. Maus, B. L. Ebert, Reversible ON- and OFF-switch chimeric antigen receptors controlled by lenalidomide. Sci Transl Med 13, eabb6295 (2021).
10. S. Carbonneau, S. Sharma, L. Peng, V. Rajan, D. Hainzl, M. Henault, C. Yang, J. Hale, J. Shulok, J. Tallarico, J. Porter, J. Brogdon, G. Dranoff, J. E. Bradner, M. Hild, C. P. Guimaraes, An IMiD-inducible degron provides reversible regulation for chimeric antigen receptor expression and activity. Cell Chem Biol (2020), doi:10.1016/j.chembiol.2020.11.012.
11. H.-S. Li, N. M. Wong, E. Tague, J. T. Ngo, A. S. Khalil, W. W. Wong, High-performance multiplex drug-gated CAR circuits. Cancer Cell (2022), doi:10.1016/j.ccell.2022.08.008.
12. Q. L. Sievers, G. Petzold, R. D. Bunker, A. Renneville, M. Slabicki, B. J. Liddicoat, W. Abdulrahman, T. Mikkelsen, B. L. Ebert, N. H. Thoma, Defining the human C2H2 zinc finger degrome targeted by thalidomide analogs through CRBN. Science 362, eaat0572 (2018).
13. K. Samejima, H. Ogawa, A. V. Ageichik, K. L. Peterson, S. H. Kaufmann, M. T. Kanemaki, W. C. Earnshaw, Auxin-induced Rapid Degradation of Inhibitor of Caspase-activated DNase (ICAD) Induces Apoptotic DNA Fragmentation, Caspase Activation, and Cell Death. J Biological Chem 289, 31617-31623 (2014).
14. M. Enari, H. Sakahira, H. Yokoyama, K. Okawa, A. Iwamatsu, S. Nagata, A caspase-activated DNase that degrades DNA during apoptosis, and its inhibitor ICAD. Nature 391, 43-50 (1998).
15. H. Sakahira, M. Enari, S. Nagata, Cleavage of CAD inhibitor in CAD activation and DNA degradation during apoptosis. Nature 391, 96-99 (1998).
16. H. Sakahira, A. Iwamatsu, S. Nagata, Specific Chaperone-like Activity of Inhibitor of Caspase-activated DNase for Caspase-activated DNase*. J Biol Chem 275, 8091-8096 (2000).
17. E.-J. Woo, Y.-G. Kim, M.-S. Kim, W.-D. Han, S. Shin, H. Robinson, S.-Y. Park, B.-H. Oh, Structural Mechanism for Inactivation and Activation of CAD/DFF40 in the Apoptotic Pathway. Mol Cell 14, 531-539 (2004).
18. T. Otomo, H. Sakahira, K. Uegaki, S. Nagata, T. Yamazaki, Structure of the heterodimeric complex between CAD domains of CAD and ICAD. Nat Struct Biol 7, 658-662 (2000).
19. B. D. Larsen, C. S. Sorensen, The caspase-activated DNase: apoptosis and beyond. Febs J 284, 1160-1170 (2017).
20. N. Chen, S. Zhou, M. Palmisano, Clinical Pharmacokinetics and Pharmacodynamics of Lenalidomide. Clin Pharmacokinet 56, 139-152 (2017).
21. J. G. Doench, N. Fusi, M. Sullender, M. Hegde, E. W. Vaimberg, K. F. Donovan, I. Smith, Z. Tothova, C. Wilen, R. Orchard, H. W. Virgin, J. Listgarten, D. E. Root, Optimized sgRNA design to maximize activity and minimize off-target effects of CRISPR-Cas9. Nat Biotechnol 34, 184-191 (2016).
22. A. S. Sperling, M. Burgess, H. Keshishian, J. A. Gasser, S. Bhatt, M. Jan, M. Slabicki, R. S. Sellar, E. C. Fink, P. G. Miller, B. J. Liddicoat, Q. L. Sievers, R. Sharma, D. N. Adams, E. A. Olesinski, M. Fulciniti, N. D. Udeshi, E. Kuhn, A. Letai, N. C. Munshi, S. A. Carr, B. L. Ebert, Patterns of substrate affinity, competition, and degradation kinetics underlie biological activity of thalidomide analogs. Blood 134, 160-170 (2019).
23. Q. L. Sievers, J. A. Gasser, G. S. Cowley, E. S. Fischer, B. L. Ebert, Genome-wide screen identifies cullin-RING ligase machinery required for lenalidomide-dependent CRL4CRBN activity. Blood 132, 1293-1303 (2018).
24. A. D. Stasi, S.-K. Tey, G. Dotti, Y. Fujita, A. Kennedy-Nasser, C. Martinez, K. Straathof, E. Liu, A. G. Durett, B. Grilley, H. Liu, C. R. Cruz, B. Savoldo, A. P. Gee, J. Schindler, R. A. Krance, H. E. Heslop, D. M. Spencer, C. M. Rooney, M. K. Brenner, Inducible Apoptosis as a Safety Switch for Adoptive Cell Therapy. New Engl J Med 365, 1673-1683 (2011).
25. C. Bonini, G. Ferrari, S. Verzeletti, P. Servida, E. Zappone, L. Ruggieri, M. Ponzoni, S. Rossini, F. Mavilio, C. Traversari, C. Bordignon, HSV-TK Gene Transfer into Donor Lymphocytes for Control of Allogeneic Graft-Versus-Leukemia. Science 276, 1719-1724 (1997).
26. B. Philip, E. Kokalaki, L. Mekkaoui, S. Thomas, K. Straathof, B. Flutter, V. Marin, T. Marafioti, R. Chakraverty, D. Linch, S. A. Quezada, K. S. Peggs, M. Pule, A highly compact epitope-based marker/suicide gene for easier and safer T-cell therapy. Blood 124, 1277-1287 (2014).
27. X. Wang, W.-C. Chang, C. W. Wong, D. Colcher, M. Sherman, J. R. Ostberg, S. J. Forman, S. R. Riddell, M. C. Jensen, A transgene-encoded cell surface polypeptide for selection, in vivo tracking, and ablation of engineered cells. Blood 118, 1255-1263 (2011).
28. V. Wiebking, J. O. Patterson, R. Martin, M. K. Chanda, C. M. Lee, W. Srifa, G. Bao, M. H. Porteus, Metabolic engineering generates a transgene-free safety switch for cell therapy. Nat Biotechnol, 1-10 (2020).
29. M. Schapira, M. F. Calabrese, A. N. Bullock, C. M. Crews, Targeted protein degradation: expanding the toolbox. Nat Rev Drug Discov 18, 949-963 (2019).
30. H. K. Chung, X. Zou, B. T. Bajar, V. R. Brand, Y. Huo, J. F. Alcudia, J. E. Ferrell, M. Z. Lin, A compact synthetic pathway rewires cancer signaling to therapeutic effector release. Science364, eaat6982 (2019).
31. L. Nissim, M.-R. Wu, E. Pery, A. Binder-Nissim, H. I. Suzuki, D. Stupp, C. Wehrspaun, Y. Tabach, P. A. Sharp, T. K. Lu, Synthetic RNA-Based Immunomodulatory Gene Circuits for Cancer Immunotherapy. Cell 171, 1138-1150.e15 (2017).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An expression construct comprising a promoter driving expression of anti-apoptotic transgene encoding a degron-tagged human inhibitor of caspase-activated DNase (ICAD) and a pro-apoptotic transgene encoding a human caspase-activated DNase (CAD), wherein the degron-tagged ICAD comprises a degron sequence at the C terminus of ICAD.

2. The expression construct of claim 1, which is multicistronic and comprises one promoter and one or more 2A or internal ribosome entry site (IRES) sequences between the anti-apoptotic transgene encoding degron-tagged ICAD and the pro-apoptotic transgene encoding CAD.

3. The expression construct of claim 2, wherein the promoter is a human elongation factor-1 alpha (EF1α) promoter.

4. The expression construct of claim 1, wherein the CAD comprises one or more mutations at residues corresponding to R11, K18, Y49, or E68 of SEQ ID NO:1.

5. The expression construct of claim 4, wherein the CAD comprises one or more mutations selected from R11A, R11W, K18A, Y49W, and E68A, and combinations thereof.

6. The expression construct of claim 5, wherein the CAD comprises mutations R11W/K18A or R11W/E68A.

7. The expression construct of claim 1, wherein the degron tag is a IKZF3 degron or dTAG.

8. The expression construct of claim 7, wherein the IKZF3 degron comprises ZFP91-IKZF3 degron (FNVLMVHKRSHTGERPLQCEICGFTCRQKGNLLRHIKLHTGEKPFKCHLCNYACQR RDAL (SEQ ID NO:3)); degron 913iK0 (FNVLMVHRRSHTGERPLQCEICGFTCRQRGNLLRHIRLHTGERPFRCHLCNYACQR RDAL (SEQ ID NO:4)); or IKZF3 degron polypeptide 130-189 (FNVLMVHKRSHTGERPFQCNQCGASFTQKGNLLRHIKLHTGEKPFKCHLCNYACQ RRDAL (SEQ ID NO:5)).

9. The expression construct of claim 1, wherein the CAD comprises a sequence at least 80% identical to SEQ ID NO:1.

10. The expression construct of claim 1, wherein the ICAD comprises a sequence at least 80% identical to SEQ ID NO:2.

11. The expression construct of claim 1, which is in a viral vector.

12. The expression construct of claim 11, wherein the viral vector is a lentivirus, adenovirus, or adeno-associated virus.

13. A cell expressing a transgene encoding a degron-tagged human inhibitor of caspase-activated DNase (ICAD), wherein the degron-tagged ICAD comprises a degron sequence at the C terminus of ICAD, and a pro-apoptotic transgene encoding human caspase-activated DNase (CAD), preferably wherein the degron-tagged ICAD and the CAD are expressed in an approximately 1:1 ratio.

14. A cell comprising the expression construct of claim 1, and optionally expressing the degron-tagged human ICAD and human CAD.

15. The cell of claim 13, wherein the cell further expresses a therapeutic transgene.

16. The cell of claim 15, wherein the therapeutic transgene is a chimeric antigen receptor (CAR).

17. The cell of claim 16, which is a T cell or a natural killer (NK) cell.

18. The cell of claim 13, which is a human cell.

19. A method of providing a cell therapy to a human subject, the method comprising:
administering to the subject the cell of claim 13.

20. The method of claim 19, further comprising administering to the subject an effective amount of a small molecule controller of the degron, wherein the small molecule controller triggers degradation of the degron-tagged anti-apoptotic ICAD protein.

21. The method of claim 20, wherein a) the degron is an IKZF3 degron and the small molecule controller comprises thalidomide or a thalidomide analog; or b) the degron is dTAG and the small molecule controller comprises dTag-13 (1-[(2S)-1-Oxo-2-(3,4,5-trimethoxyphenyl)butyl]-(2S)-2-piperidinecarboxylate (1R)-3-(3,4-dimethoxyphenyl)-1-[2-[2-[[6-[[2-(2,6-dioxo-3-piperidinyl)-2,3-dihydro-1,3-dioxo-1H-isoindol-4-yl]oxy]hexyl]amino]-2-oxoethoxy]phenyl]propyl ester.


SEQUENCE LISTING (36 sequences)

SEQ ID NO: 1 - protein, 338 aa, Homo sapiens
MLQKPKSVKLRALRSPRKFGVAGRSCQEVLRKGCLRFQLPERGSRLCLYEDGTELTEDYFPSVPDNAELVLLTLGQAWQGYVSDIRRFLSAFHEPQVGLIQAAQQLLCDEQAPQRQRLLADLLHNVSQNIAAETRAEDPPWFEGLESRFQSKSGYLRYSCESRIRSYLREVSSYPSTVGAEAQEEFLRVLGSMCQRLRSMQYNGSYFDRGAKGGSRLCTPEGWFSCQGPFDMDSCLSRHSINPYSNRESRILFSTWNLDHIIEKKRTIIPTLVEAIKEQDGREVDWEYFYGLLFTSENLKLVHIVCHKKTTHKLNCDPSRIYKPQTRLKRKQPVRKRQ

SEQ ID NO: 2 - protein, 331 aa, Homo sapiens
MEVTGDAGVPESGEIRTLKPCLLRRNYSREQHGVAASCLEDLRSKACDILAIDKSLTPVTLVLAEDGTIVDDDDYFLCLPSNTKFVALASNEKWAYNNSDGGTAWISQESFDVDETDSGAGLKWKNVARQLKEDLSSIILLSEEDLQMLVDAPCSDLAQELRQSCATVQRLQHTLQQVLDQREEVRQSKQLLQLYLQALEKEGSLLSKQEESKAAFGEEVDAVDTGISRETSSDVALASHILTALREKQAPELSLSSQDLELVTKEDPKALAVALNWDIKKTETVQEACERELALRLQQTQSLHSLRSISASKASPPGDLQNPKRARQDPT

SEQ ID NO: 3 - protein, 60 aa, synthetic construct
FNVLMVHKRSHTGERPLQCEICGFTCRQKGNLLRHIKLHTGEKPFKCHLCNYACQRRDAL

SEQ ID NO: 4 - protein, 60 aa, synthetic construct
FNVLMVHRRSHTGERPLQCEICGFTCRQRGNLLRHIRLHTGERPFRCHLCNYACQRRDAL

SEQ ID NO: 5 - protein, 60 aa, synthetic construct
FNVLMVHKRSHTGERPFQCNQCGASFTQKGNLLRHIKLHTGEKPFKCHLCNYACQRRDAL

SEQ ID NO: 6 - protein, 8 aa, synthetic construct
DXEXNPGP

SEQ ID NO: 7 - DNA, 699 bp, synthetic construct
  source 1..699: mol_type = other DNA
atgtctcagtcaaatagggaactcgtggtcgattttttgagctacaaacttagtcaaaaaggatatagctggtctcagttttccgatgttgaggagaacagaacggaagcgcccgaggggactgaaagcgaaatggaaactccgtcagccatcaacggtaatccttcctggcacctcgctgattcacctgcagtcaatggagcaacaggacattcatcatcactcgacgcaagagaagttattccgatggcggctgtaaagcaggcattgcgggaagcgggtgacgaatttgagttgaggtacaggagggctttctctgacctgactagccaactgcatattacgccaggcactgcttaccagagttttgaacaagtggtaaatgaactctttagagatggggtcaactggggacggatagttgcttttttcagcttcggcggagccttgtgtgtagagagcgtggacaaggaaatgcaggttctcgtcagcaggattgcagcctggatggcgacttacttgaatgatcacctcgaaccatggattcaagaaaatggtggttgggacacgttcgtggaactctacggcaataatgccgcagcagaaagccgcaaaggccaagaaaggttcaaccgatggtttcttaccggaatgacagtcgctggcgttgtgctccttgggtcactcttttcacggaag

SEQ ID NO: 8 - protein, 233 aa, synthetic construct
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 9 - DNA, 594 bp, synthetic construct
  source 1..594: mol_type = other DNA
atggcaaagcagccctcagatgtgtcttccgaatgcgacagggaaggacgccagcttcagcctgctgagcggccccctcaactcagaccgggagcgccaacttcactccaaacagaaccccaaggcaatcccgagggtaaccacggaggggaaggtgattcatgtccgcatggttccccacagggaccgttggcgccaccggcgtcaccaggtcctttcgcaactaggtcaccgctcttcatcttcatgcggcgatcttcccttttgagtcgctcaagcagcggttacttcagcttcgataccgaccgatcaccggcaccaatgagctgtgacaaatcaacccaaaccccttctccaccttgccaagcctttaaccactacctgtcagctatggcatcaatgcgacaagctgagccagctgatatgcgcccggaaatatggatagcacaagaattgcggcgaatcggcgacgagtttaatgcttactacgcgcgccgcgtgtttctgaataattaccaagccgctgaggaccaccccagaatggtcattcttaggctcctgcgctacatagtcaggttggtgtggcggatgcat

SEQ ID NO: 10 - protein, 198 aa, synthetic construct
MAKQPSDVSSECDREGRQLQPAERPPQLRPGAPTSLQTEPQGNPEGNHGGEGDSCPHGSPQGPLAPPASPGPFATRSPLFIFMRRSSLLSRSSSGYFSFDTDRSPAPMSCDKSTQTPSPPCQAFNHYLSAMASMRQAEPADMRPEIWIAQELRRIGDEFNAYYARRVFLNNYQAAEDHPRMVILRLLRYIVRLVWRMH

SEQ ID NO: 11 - DNA, 75 bp, synthetic construct
  source 1..75: mol_type = other DNA
gaggagcaggccgcaagagaaatcggagcacaacttcggcgaatggctgacgacttgaacgcacagtatgagcgg

SEQ ID NO: 12 - protein, 25 aa, synthetic construct
EEQAAREIGAQLRRMADDLNAQYER

SEQ ID NO: 13 - DNA, 393 bp, synthetic construct
  source 1..393: mol_type = other DNA
atgaaattcgggatgggtagtgcccaagcctgtccctgccaagttccgcgcgcagcgtcaacgacatgggtgccctgccagatatgcggcccccaaccttctctcagcctcgctgaacaacatttggagagtcctgtgcctagtgctcctggcgcgttggcgggaggtcctacccaagcagctcctggggtacgaggtgaagaagagcaagccgcccgagaaataggcgcccagttgagaagaatggctgatgacctcaacgcacagtacgaaagacgacgacaagaggaacaacagcgccaccggccttcaccatggcgagttctgtacaatcttattatggggcttcttccgcttcctcggggccacagagcccctgagatggagcctaac

SEQ ID NO: 14 - protein, 131 aa, synthetic construct
MKFGMGSAQACPCQVPRAASTTWVPCQICGPQPSLSLAEQHLESPVPSAPGALAGGPTQAAPGVRGEEEQAAREIGAQLRRMADDLNAQYERRRQEEQQRHRPSPWRVLYNLIMGLLPLPRGHRAPEMEPN

SEQ ID NO: 15 - DNA, 2289 bp, synthetic construct
  source 1..2289: mol_type = other DNA
atggaagtgactggtgatgccggtgtcccagagagcggcgagatcaggactcttaaaccatgcctgcttaggcgaaactacagccgagagcaacatggtgttgctgctagttgcttggaagacctgcgatccaaggcgtgtgacatcttggcaattgataagtcactcacgccggttactttggtgcttgccgaagacggcacaatagtagacgacgacgattacttcctctgccttccgtcaaacacgaagtttgttgcacttgcgagtaatgagaaatgggcttacaacaattcagatggtggcaccgcctggattagccaggaatctttcgacgtagatgaaacagactctggggccggtctcaaatggaagaacgttgcccggcagcttaaagaagatctgtctagcataatcctgctctctgaggaggatctgcagatgctggtggatgcgccctgtagtgatttggcccaagaacttcgacaaagttgtgcaactgtacaacggctccaacacacattgcagcaggtgctggaccagcgagaggaggtgaggcagagcaagcaacttcttcaactctacctccaagctcttgagaaagagggctcattgctctcaaaacaagaagagtctaaagcagcgtttggtgaggaagtcgacgctgtagacactggaatttcaagagagactagttccgatgttgcgcttgctagtcatattcttacagctctgagagagaagcaagctccagaactctctctcagctcccaagacctcgaacttgttactaaagaagacccgaaggcgctggcagtcgctcttaactgggacatcaagaagacagagacagttcaagaggcatgtgaacgggagctggcattgcgcctccaacaaactcaatccctgcattccctgcggtccataagcgcctcaaaagcaagccctccaggggatctgcagaatcctaagagggcgcggcaagatcccacgggcagtggctcgggctcggggtccggcggattcaatgtcttaatggttcataagcgaagccatactggtgaacgcccattgcagtgcgaaatatgcggctttacctgccgccagaaaggtaacctcctccgccacattaaactgcacacaggggaaaaaccttttaagtgtcacctctgcaactatgcatgccaaagaagagatgcgctctccggaggcggcggagagggcagaggaagtcttctaacatgcggtgacgtggaggagaatcccggccctaggatgcttcagaaacccaagagtgtgaagttgcgggccttgcgctcacctagaaagtttggagtagctggacgaagttgtcaggaggtgcttcggaaaggctgcctgcgattccagctgcccgaaagaggttctcggttgtgtttgtatgaggatggaaccgaactgacagaggactattttccctccgtacctgacaatgcggagttggtacttttgacccttggtcaagcttggcagggatatgtctctgatataaggcgcttcctctcagctttccacgaaccccaggtggggctcatacaagcagcgcagcagctgctgtgcgacgagcaagcaccccaacgacaacgactgttggcggacttgttgcataacgtatcccaaaatattgcggcggagacacgcgccgaggaccctccatggttcgagggacttgaaagtcgctttcagtccaaaagcggatatcttcgatactcatgtgaatctcgcattagaagctaccttagagaagtctctagttatccgagtactgtcggcgcagaggcacaagaggagttcttgagagtcttgggctccatgtgtcaacggttgcggtccatgcaatacaatggcagctattttgaccggggcgctaaaggtggctctcggctttgcacacccgagggctggttctcttgccagggccccttcgacatggactcttgcctttcacggcactctatcaatccatactccaatcgggaatccaggattctcttcagcacctggaatctcgatcatataatagaaaagaaacggactattataccaactctcgtagaagccataaaggaacaggatgggcgggaagtagattgggaatatttctatggcctccttttcacttccgaaaacttgaaactggtgcatatagtttgccataagaagaccacccataagttgaactgcgatccctcaagaatatataaacctcagaccaggctcaagcgaaagcagcctgtccgaaaacgacag

SEQ ID NO: 16 - protein, 763 aa, synthetic construct
MEVTGDAGVPESGEIRTLKPCLLRRNYSREQHGVAASCLEDLRSKACDILAIDKSLTPVTLVLAEDGTIVDDDDYFLCLPSNTKFVALASNEKWAYNNSDGGTAWISQESFDVDETDSGAGLKWKNVARQLKEDLSSIILLSEEDLQMLVDAPCSDLAQELRQSCATVQRLQHTLQQVLDQREEVRQSKQLLQLYLQALEKEGSLLSKQEESKAAFGEEVDAVDTGISRETSSDVALASHILTALREKQAPELSLSSQDLELVTKEDPKALAVALNWDIKKTETVQEACERELALRLQQTQSLHSLRSISASKASPPGDLQNPKRARQDPTGSGSGSGSGGFNVLMVHKRSHTGERPLQCEICGFTCRQKGNLLRHIKLHTGEKPFKCHLCNYACQRRDALSGGGGEGRGSLLTCGDVEENPGPRMLQKPKSVKLRALRSPRKFGVAGRSCQEVLRKGCLRFQLPERGSRLCLYEDGTELTEDYFPSVPDNAELVLLTLGQAWQGYVSDIRRFLSAFHEPQVGLIQAAQQLLCDEQAPQRQRLLADLLHNVSQNIAAETRAEDPPWFEGLESRFQSKSGYLRYSCESRIRSYLREVSSYPSTVGAEAQEEFLRVLGSMCQRLRSMQYNGSYFDRGAKGGSRLCTPEGWFSCQGPFDMDSCLSRHSINPYSNRESRILFSTWNLDHIIEKKRTIIPTLVEAIKEQDGREVDWEYFYGLLFTSENLKLVHIVCHKKTTHKLNCDPSRIYKPQTRLKRKQPVRKRQ

SEQ ID NO: 17 - DNA, 2289 bp, synthetic construct
  source 1..2289: mol_type = other DNA
atggaagtgactggtgatgccggtgtcccagagagcggcgagatcaggactcttaaaccatgcctgcttaggcgaaactacagccgagagcaacatggtgttgctgctagttgcttggaagacctgcgatccaaggcgtgtgacatcttggcaattgataagtcactcacgccggttactttggtgcttgccgaagacggcacaatagtagacgacgacgattacttcctctgccttccgtcaaacacgaagtttgttgcacttgcgagtaatgagaaatgggcttacaacaattcagatggtggcaccgcctggattagccaggaatctttcgacgtagatgaaacagactctggggccggtctcaaatggaagaacgttgcccggcagcttaaagaagatctgtctagcataatcctgctctctgaggaggatctgcagatgctggtggatgcgccctgtagtgatttggcccaagaacttcgacaaagttgtgcaactgtacaacggctccaacacacattgcagcaggtgctggaccagcgagaggaggtgaggcagagcaagcaacttcttcaactctacctccaagctcttgagaaagagggctcattgctctcaaaacaagaagagtctaaagcagcgtttggtgaggaagtcgacgctgtagacactggaatttcaagagagactagttccgatgttgcgcttgctagtcatattcttacagctctgagagagaagcaagctccagaactctctctcagctcccaagacctcgaacttgttactaaagaagacccgaaggcgctggcagtcgctcttaactgggacatcaagaagacagagacagttcaagaggcatgtgaacgggagctggcattgcgcctccaacaaactcaatccctgcattccctgcggtccataagcgcctcaaaagcaagccctccaggggatctgcagaatcctaagagggcgcggcaagatcccacgggcagtggctcgggctcggggtccggcggattcaatgtcttaatggttcataagcgaagccatactggtgaacgcccattgcagtgcgaaatatgcggctttacctgccgccagaaaggtaacctcctccgccacattaaactgcacacaggggaaaaaccttttaagtgtcacctctgcaactatgcatgccaaagaagagatgcgctctccggaggcggcggagagggcagaggaagtcttctaacatgcggtgacgtggaggagaatcccggccctaggatgctccagaagcccaagagcgtgaagctgcgggccctgcgcagcccgaggaagttcggcgtggctggccggagctgccaggaggtgctgcgcaagggctgtctccgcttccagctccctgagcgcggttcccggctgtgcctgtacgaggatggcacggagctgacggaagattacttccccagtgttcccgacaacgccgagctggtgctgctcaccttgggccaggcctggcagggctatgtgagcgacatcaggcgcttcctcagtgcatttcacgagccacaggtggggctcatccaggccgcccagcagctgctgtgtgatgagcaggccccacagaggcagaggctgctggctgacctcctgcacaacgtcagccagaacatcgcggccgagacccgggctgaggacccgccgtggtttgaaggcttggagtcccgatttcagagcaagtctggctatctgagatacagctgtgagagccggatccggagttacctgagggaggtgagctcctacccctccacggtgggtgcggaggctcaggaggaattcctgcgggtcctcggctccatgtgccagaggctccggtccatgcagtacaatggcagctacttcgacagaggagccaagggcggcagccgcctctgcacaccggaaggctggttctcctgccagggtccctttgacatggacagctgcttatcaagacactccatcaacccctacagtaacagggagagcaggatcctcttcagcacctggaacctggatcacataatagaaaagaaacgcaccatcattcctacactggtggaagcaattaaggaacaagatggaagagaagtggactgggagtatttttatggcctgctttttacctcagagaacctaaaactagtgcacattgtctgccataagaaaaccacccacaagctcaactgtgacccaagcagaatctacaaaccccagacaaggttgaagcggaagcagcctgtgcggaaacgccag

SEQ ID NO: 18 - protein, 763 aa, synthetic construct; identical to SEQ ID NO: 16

SEQ ID NO: 19 - DNA, 2289 bp, synthetic construct
  source 1..2289: mol_type = other DNA
atggaagtgactggtgatgccggtgtcccagagagcggcgagatcaggactcttaaaccatgcctgcttaggcgaaactacagccgagagcaacatggtgttgctgctagttgcttggaagacctgcgatccaaggcgtgtgacatcttggcaattgataagtcactcacgccggttactttggtgcttgccgaagacggcacaatagtagacgacgacgattacttcctctgccttccgtcaaacacgaagtttgttgcacttgcgagtaatgagaaatgggcttacaacaattcagatggtggcaccgcctggattagccaggaatctttcgacgtagatgaaacagactctggggccggtctcaaatggaagaacgttgcccggcagcttaaagaagatctgtctagcataatcctgctctctgaggaggatctgcagatgctggtggatgcgccctgtagtgatttggcccaagaacttcgacaaagttgtgcaactgtacaacggctccaacacacattgcagcaggtgctggaccagcgagaggaggtgaggcagagcaagcaacttcttcaactctacctccaagctcttgagaaagagggctcattgctctcaaaacaagaagagtctaaagcagcgtttggtgaggaagtcgacgctgtagacactggaatttcaagagagactagttccgatgttgcgcttgctagtcatattcttacagctctgagagagaagcaagctccagaactctctctcagctcccaagacctcgaacttgttactaaagaagacccgaaggcgctggcagtcgctcttaactgggacatcaagaagacagagacagttcaagaggcatgtgaacgggagctggcattgcgcctccaacaaactcaatccctgcattccctgcggtccataagcgcctcaaaagcaagccctccaggggatctgcagaatcctaagagggcgcggcaagatcccacgggcagtggctcgggctcggggtccggcggattcaatgtcttaatggttcataagcgaagccatactggtgaacgcccattgcagtgcgaaatatgcggctttacctgccgccagaaaggtaacctcctccgccacattaaactgcacacaggggaaaaaccttttaagtgtcacctctgcaactatgcatgccaaagaagagatgcgctctccggaggcggcggagagggcagaggaagtcttctaacatgcggtgacgtggaggagaatcccggccctaggatgctccagaagcccaagagcgtgaagctgtgggccctgcgcagcccgaggaagttcggcgtggctggccggagctgccaggaggtgctgcgcaagggctgtctccgcttccagctccctgagcgcggttcccggctgtgcctgtacgaggatggcacggagctgacggaagattacttccccagtgttcccgacaacgccgagctggtgctgctcaccttgggccaggcctggcagggctatgtgagcgacatcaggcgcttcctcagtgcatttcacgagccacaggtggggctcatccaggccgcccagcagctgctgtgtgatgagcaggccccacagaggcagaggctgctggctgacctcctgcacaacgtcagccagaacatcgcggccgagacccgggctgaggacccgccgtggtttgaaggcttggagtcccgatttcagagcaagtctggctatctgagatacagctgtgagagccggatccggagttacctgagggaggtgagctcctacccctccacggtgggtgcggaggctcaggaggaattcctgcgggtcctcggctccatgtgccagaggctccggtccatgcagtacaatggcagctacttcgacagaggagccaagggcggcagccgcctctgcacaccggaaggctggttctcctgccagggtccctttgacatggacagctgcttatcaagacactccatcaacccctacagtaacagggagagcaggatcctcttcagcacctggaacctggatcacataatagaaaagaaacgcaccatcattcctacactggtggaagcaattaaggaacaagatggaagagaagtggactgggagtatttttatggcctgctttttacctcagagaacctaaaactagtgcacattgtctgccataagaaaaccacccacaagctcaactgtgacccaagcagaatctacaaaccccagacaaggttgaagcggaagcagcctgtgcggaaacgccag

SEQ ID NO: 20 - protein, 763 aa, synthetic construct
MEVTGDAGVPESGEIRTLKPCLLRRNYSREQHGVAASCLEDLRSKACDILAIDKSLTPVTLVLAEDGTIVDDDDYFLCLPSNTKFVALASNEKWAYNNSDGGTAWISQESFDVDETDSGAGLKWKNVARQLKEDLSSIILLSEEDLQMLVDAPCSDLAQELRQSCATVQRLQHTLQQVLDQREEVRQSKQLLQLYLQALEKEGSLLSKQEESKAAFGEEVDAVDTGISRETSSDVALASHILTALREKQAPELSLSSQDLELVTKEDPKALAVALNWDIKKTETVQEACERELALRLQQTQSLHSLRSISASKASPPGDLQNPKRARQDPTGSGSGSGSGGFNVLMVHKRSHTGERPLQCEICGFTCRQKGNLLRHIKLHTGEKPFKCHLCNYACQRRDALSGGGGEGRGSLLTCGDVEENPGPRMLQKPKSVKLWALRSPRKFGVAGRSCQEVLRKGCLRFQLPERGSRLCLYEDGTELTEDYFPSVPDNAELVLLTLGQAWQGYVSDIRRFLSAFHEPQVGLIQAAQQLLCDEQAPQRQRLLADLLHNVSQNIAAETRAEDPPWFEGLESRFQSKSGYLRYSCESRIRSYLREVSSYPSTVGAEAQEEFLRVLGSMCQRLRSMQYNGSYFDRGAKGGSRLCTPEGWFSCQGPFDMDSCLSRHSINPYSNRESRILFSTWNLDHIIEKKRTIIPTLVEAIKEQDGREVDWEYFYGLLFTSENLKLVHIVCHKKTTHKLNCDPSRIYKPQTRLKRKQPVRKRQ

SEQ ID NO: 21 - DNA, 1950 bp, synthetic construct
  source 1..1950: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggcggcggcggctccggcggcggcggctccatggaagtgactggtgatgccggtgtcccagagagcggcgagatcaggactcttaaaccatgcctgcttaggcgaaactacagccgagagcaacatggtgttgctgctagttgcttggaagacctgcgatccaaggcgtgtgacatcttggcaattgataagtcactcacgccggttactttggtgcttgccgaagacggcacaatagtagacgacgacgattacttcctctgccttccgtcaaacacgaagtttgttgcacttgcgagtaatgagaaatgggcttacaacaattcagatggtggcaccgcctggattagccaggaatctttcgacgtagatgaaacagactctggggccggtctcaaatggaagaacgttgcccggcagcttaaagaagatctgtctagcataatcctgctctctgaggaggatctgcagatgctggtggatgcgccctgtagtgatttggcccaagaacttcgacaaagttgtgcaactgtacaacggctccaacacacattgcagcaggtgctggaccagcgagaggaggtgaggcagagcaagcaacttcttcaactctacctccaagctcttgagaaagagggctcattgctctcaaaacaagaagagtctaaagcagcgtttggtgaggaagtcgacgctgtagacactggaatttcaagagagactagttccgatgttgcgcttgctagtcatattcttacagctctgagagagaagcaagctccagaactctctctcagctcccaagacctcgaacttgttactaaagaagacccgaaggcgctggcagtcgctcttaactgggacatcaagaagacagagacagttcaagaggcatgtgaacgggagctggcattgcgcctccaacaaactcaatccctgcattccctgcggtccataagcgcctcaaaagcaagccctccaggggatctgcagaatcctaagagggcgcggcaagatcccacgggcagtggctcgggctcggggtccggcggattcaatgtcttaatggttcataagcgaagccatactggtgaacgcccattgcagtgcgaaatatgcggctttacctgccgccagaaaggtaacctcctccgccacattaaactgcacacaggggaaaaaccttttaagtgtcacctctgcaactatgcatgccaaagaagagatgcgctc

SEQ ID NO: 22 - protein, 650 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGGGGSGGGGSMEVTGDAGVPESGEIRTLKPCLLRRNYSREQHGVAASCLEDLRSKACDILAIDKSLTPVTLVLAEDGTIVDDDDYFLCLPSNTKFVALASNEKWAYNNSDGGTAWISQESFDVDETDSGAGLKWKNVARQLKEDLSSIILLSEEDLQMLVDAPCSDLAQELRQSCATVQRLQHTLQQVLDQREEVRQSKQLLQLYLQALEKEGSLLSKQEESKAAFGEEVDAVDTGISRETSSDVALASHILTALREKQAPELSLSSQDLELVTKEDPKALAVALNWDIKKTETVQEACERELALRLQQTQSLHSLRSISASKASPPGDLQNPKRARQDPTGSGSGSGSGGFNVLMVHKRSHTGERPLQCEICGFTCRQKGNLLRHIKLHTGEKPFKCHLCNYACQRRDAL

SEQ ID NO: 23 - DNA, 2289 bp, synthetic construct
  source 1..2289: mol_type = other DNA
atggaagtgactggtgatgccggtgtcccagagagcggcgagatcaggactcttaaaccatgcctgcttaggcgaaactacagccgagagcaacatggtgttgctgctagttgcttggaagacctgcgatccaaggcgtgtgacatcttggcaattgataagtcactcacgccggttactttggtgcttgccgaagacggcacaatagtagacgacgacgattacttcctctgccttccgtcaaacacgaagtttgttgcacttgcgagtaatgagaaatgggcttacaacaattcagatggtggcaccgcctggattagccaggaatctttcgacgtagatgaaacagactctggggccggtctcaaatggaagaacgttgcccggcagcttaaagaagatctgtctagcataatcctgctctctgaggaggatctgcagatgctggtggatgcgccctgtagtgatttggcccaagaacttcgacaaagttgtgcaactgtacaacggctccaacacacattgcagcaggtgctggaccagcgagaggaggtgaggcagagcaagcaacttcttcaactctacctccaagctcttgagaaagagggctcattgctctcaaaacaagaagagtctaaagcagcgtttggtgaggaagtcgacgctgtagacactggaatttcaagagagactagttccgatgttgcgcttgctagtcatattcttacagctctgagagagaagcaagctccagaactctctctcagctcccaagacctcgaacttgttactaaagaagacccgaaggcgctggcagtcgctcttaactgggacatcaagaagacagagacagttcaagaggcatgtgaacgggagctggcattgcgcctccaacaaactcaatccctgcattccctgcggtccataagcgcctcaaaagcaagccctccaggggatctgcagaatcctaagagggcgcggcaagatcccacgggcagtggctcgggctcggggtccggcggattcaatgtcttaatggttcataagcgaagccatactggtgaacgcccattgcagtgcgaaatatgcggctttacctgccgccagaaaggtaacctcctccgccacattaaactgcacacaggggaaaaaccttttaagtgtcacctctgcaactatgcatgccaaagaagagatgcgctctccggaggcggcggagagggcagaggaagtcttctaacatgcggtgacgtggaggagaatcccggccctaggatgctccagaagcccaagagcgtgaagctggccgccctgcgcagcccgaggaagttcggcgtggctggccggagctgccaggaggtgctgcgcaagggctgtctccgcttccagctccctgagcgcggttcccggctgtgcctgtacgaggatggcacggagctgacggaagattacttccccagtgttcccgacaacgccgagctggtgctgctcaccttgggccaggcctggcagggctatgtgagcgacatcaggcgcttcctcagtgcatttcacgagccacaggtggggctcatccaggccgcccagcagctgctgtgtgatgagcaggccccacagaggcagaggctgctggctgacctcctgcacaacgtcagccagaacatcgcggccgagacccgggctgaggacccgccgtggtttgaaggcttggagtcccgatttcagagcaagtctggctatctgagatacagctgtgagagccggatccggagttacctgagggaggtgagctcctacccctccacggtgggtgcggaggctcaggaggaattcctgcgggtcctcggctccatgtgccagaggctccggtccatgcagtacaatggcagctacttcgacagaggagccaagggcggcagccgcctctgcacaccggaaggctggttctcctgccagggtccctttgacatggacagctgcttatcaagacactccatcaacccctacagtaacagggagagcaggatcctcttcagcacctggaacctggatcacataatagaaaagaaacgcaccatcattcctacactggtggaagcaattaaggaacaagatggaagagaagtggactgggagtatttttatggcctgctttttacctcagagaacctaaaactagtgcacattgtctgccataagaaaaccacccacaagctcaactgtgacccaagcagaatctacaaaccccagacaaggttgaagcggaagcagcctgtgcggaaacgccag

SEQ ID NO: 24 - protein, 763 aa, synthetic construct
MEVTGDAGVPESGEIRTLKPCLLRRNYSREQHGVAASCLEDLRSKACDILAIDKSLTPVTLVLAEDGTIVDDDDYFLCLPSNTKFVALASNEKWAYNNSDGGTAWISQESFDVDETDSGAGLKWKNVARQLKEDLSSIILLSEEDLQMLVDAPCSDLAQELRQSCATVQRLQHTLQQVLDQREEVRQSKQLLQLYLQALEKEGSLLSKQEESKAAFGEEVDAVDTGISRETSSDVALASHILTALREKQAPELSLSSQDLELVTKEDPKALAVALNWDIKKTETVQEACERELALRLQQTQSLHSLRSISASKASPPGDLQNPKRARQDPTGSGSGSGSGGFNVLMVHKRSHTGERPLQCEICGFTCRQKGNLLRHIKLHTGEKPFKCHLCNYACQRRDALSGGGGEGRGSLLTCGDVEENPGPRMLQKPKSVKLAALRSPRKFGVAGRSCQEVLRKGCLRFQLPERGSRLCLYEDGTELTEDYFPSVPDNAELVLLTLGQAWQGYVSDIRRFLSAFHEPQVGLIQAAQQLLCDEQAPQRQRLLADLLHNVSQNIAAETRAEDPPWFEGLESRFQSKSGYLRYSCESRIRSYLREVSSYPSTVGAEAQEEFLRVLGSMCQRLRSMQYNGSYFDRGAKGGSRLCTPEGWFSCQGPFDMDSCLSRHSINPYSNRESRILFSTWNLDHIIEKKRTIIPTLVEAIKEQDGREVDWEYFYGLLFTSENLKLVHIVCHKKTTHKLNCDPSRIYKPQTRLKRKQPVRKRQ

SEQ ID NO: 25 - DNA, 2289 bp, synthetic construct
  source 1..2289: mol_type = other DNA
atggaagtgactggtgatgccggtgtcccagagagcggcgagatcaggactcttaaaccatgcctgcttaggcgaaactacagccgagagcaacatggtgttgctgctagttgcttggaagacctgcgatccaaggcgtgtgacatcttggcaattgataagtcactcacgccggttactttggtgcttgccgaagacggcacaatagtagacgacgacgattacttcctctgccttccgtcaaacacgaagtttgttgcacttgcgagtaatgagaaatgggcttacaacaattcagatggtggcaccgcctggattagccaggaatctttcgacgtagatgaaacagactctggggccggtctcaaatggaagaacgttgcccggcagcttaaagaagatctgtctagcataatcctgctctctgaggaggatctgcagatgctggtggatgcgccctgtagtgatttggcccaagaacttcgacaaagttgtgcaactgtacaacggctccaacacacattgcagcaggtgctggaccagcgagaggaggtgaggcagagcaagcaacttcttcaactctacctccaagctcttgagaaagagggctcattgctctcaaaacaagaagagtctaaagcagcgtttggtgaggaagtcgacgctgtagacactggaatttcaagagagactagttccgatgttgcgcttgctagtcatattcttacagctctgagagagaagcaagctccagaactctctctcagctcccaagacctcgaacttgttactaaagaagacccgaaggcgctggcagtcgctcttaactgggacatcaagaagacagagacagttcaagaggcatgtgaacgggagctggcattgcgcctccaacaaactcaatccctgcattccctgcggtccataagcgcctcaaaagcaagccctccaggggatctgcagaatcctaagagggcgcggcaagatcccacgggcagtggctcgggctcggggtccggcggattcaatgtcttaatggttcataagcgaagccatactggtgaacgcccattgcagtgcgaaatatgcggctttacctgccgccagaaaggtaacctcctccgccacattaaactgcacacaggggaaaaaccttttaagtgtcacctctgcaactatgcatgccaaagaagagatgcgctctccggaggcggcggagagggcagaggaagtcttctaacatgcggtgacgtggaggagaatcccggccctaggatgctccagaagcccaagagcgtgaagctgagggccctgcgcagcccgaggaagttcggcgtggctggccggagctgccaggaggtgctgcgcaagggctgtctccgcttccagctccctgagcgcggttcccggctgtgcctgtgggaggatggcacggagctgacggaagattacttccccagtgttcccgacaacgccgagctggtgctgctcaccttgggccaggcctggcagggctatgtgagcgacatcaggcgcttcctcagtgcatttcacgagccacaggtggggctcatccaggccgcccagcagctgctgtgtgatgagcaggccccacagaggcagaggctgctggctgacctcctgcacaacgtcagccagaacatcgcggccgagacccgggctgaggacccgccgtggtttgaaggcttggagtcccgatttcagagcaagtctggctatctgagatacagctgtgagagccggatccggagttacctgagggaggtgagctcctacccctccacggtgggtgcggaggctcaggaggaattcctgcgggtcctcggctccatgtgccagaggctccggtccatgcagtacaatggcagctacttcgacagaggagccaagggcggcagccgcctctgcacaccggaaggctggttctcctgccagggtccctttgacatggacagctgcttatcaagacactccatcaacccctacagtaacagggagagcaggatcctcttcagcacctggaacctggatcacataatagaaaagaaacgcaccatcattcctacactggtggaagcaattaaggaacaagatggaagagaagtggactgggagtatttttatggcctgctttttacctcagagaacctaaaactagtgcacattgtctgccataagaaaaccacccacaagctcaactgtgacccaagcagaatctacaaaccccagacaaggttgaagcggaagcagcctgtgcggaaacgccag

SEQ ID NO: 26 - protein, 763 aa, synthetic construct
MEVTGDAGVPESGEIRTLKPCLLRRNYSREQHGVAASCLEDLRSKACDILAIDKSLTPVTLVLAEDGTIVDDDDYFLCLPSNTKFVALASNEKWAYNNSDGGTAWISQESFDVDETDSGAGLKWKNVARQLKEDLSSIILLSEEDLQMLVDAPCSDLAQELRQSCATVQRLQHTLQQVLDQREEVRQSKQLLQLYLQALEKEGSLLSKQEESKAAFGEEVDAVDTGISRETSSDVALASHILTALREKQAPELSLSSQDLELVTKEDPKALAVALNWDIKKTETVQEACERELALRLQQTQSLHSLRSISASKASPPGDLQNPKRARQDPTGSGSGSGSGGFNVLMVHKRSHTGERPLQCEICGFTCRQKGNLLRHIKLHTGEKPFKCHLCNYACQRRDALSGGGGEGRGSLLTCGDVEENPGPRMLQKPKSVKLRALRSPRKFGVAGRSCQEVLRKGCLRFQLPERGSRLCLWEDGTELTEDYFPSVPDNAELVLLTLGQAWQGYVSDIRRFLSAFHEPQVGLIQAAQQLLCDEQAPQRQRLLADLLHNVSQNIAAETRAEDPPWFEGLESRFQSKSGYLRYSCESRIRSYLREVSSYPSTVGAEAQEEFLRVLGSMCQRLRSMQYNGSYFDRGAKGGSRLCTPEGWFSCQGPFDMDSCLSRHSINPYSNRESRILFSTWNLDHIIEKKRTIIPTLVEAIKEQDGREVDWEYFYGLLFTSENLKLVHIVCHKKTTHKLNCDPSRIYKPQTRLKRKQPVRKRQ

SEQ ID NO: 27 - DNA, 2289 bp, synthetic construct; identical to SEQ ID NO: 23
  source 1..2289: mol_type = other DNA

SEQ ID NO: 28 - protein, 763 aa, synthetic construct; identical to SEQ ID NO: 24

SEQ ID NO: 29 - DNA, 2451 bp, synthetic construct
  source 1..2451: mol_type = other DNA
atggaagtgactggtgatgccggtgtcccagagagcggcgagatcaggactcttaaaccatgcctgcttaggcgaaactacagccgagagcaacatggtgttgctgctagttgcttggaagacctgcgatccaaggcgtgtgacatcttggcaattgataagtcactcacgccggttactttggtgcttgccgaagacggcacaatagtagacgacgacgattacttcctctgccttccgtcaaacacgaagtttgttgcacttgcgagtaatgagaaatgggcttacaacaattcagatggtggcaccgcctggattagccaggaatctttcgacgtagatgaaacagactctggggccggtctcaaatggaagaacgttgcccggcagcttaaagaagatctgtctagcataatcctgctctctgaggaggatctgcagatgctggtggatgcgccctgtagtgatttggcccaagaacttcgacaaagttgtgcaactgtacaacggctccaacacacattgcagcaggtgctggaccagcgagaggaggtgaggcagagcaagcaacttcttcaactctacctccaagctcttgagaaagagggctcattgctctcaaaacaagaagagtctaaagcagcgtttggtgaggaagtcgacgctgtagacactggaatttcaagagagactagttccgatgttgcgcttgctagtcatattcttacagctctgagagagaagcaagctccagaactctctctcagctcccaagacctcgaacttgttactaaagaagacccgaaggcgctggcagtcgctcttaactgggacatcaagaagacagagacagttcaagaggcatgtgaacgggagctggcattgcgcctccaacaaactcaatccctgcattccctgcggtccataagcgcctcaaaagcaagccctccaggggatctgcagaatcctaagagggcgcggcaagatcccacgggcagtggctcgggctcggggtccggcggattcaatgtcttaatggttcataagcgaagccatactggtgaacgcccattgcagtgcgaaatatgcggctttacctgccgccagaaaggtaacctcctccgccacattaaactgcacacaggggaaaaaccttttaagtgtcacctctgcaactatgcatgccaaagaagagatgcgctctccggaggcggcggagagggcagaggaagtcttctaacatgcggtgacgtggaggagaatcccggccctaggatgctccagaagcccaagagcgtgaagctgtgggccctgcgcagcccgaggaagttcggcgtggctggccggagctgccaggaggtgctgcgcaagggctgtctccgcttccagctccctgagcgcggttcccggctgtgcctgtacgaggatggcacggagctgacggaagattacttccccagtgttcccgacaacgccgccctggtgctgctcaccttgggccaggcctggcagggctatgtgagcgacatcaggcgcttcctcagtgcatttcacgagccacaggtggggctcatccaggccgcccagcagctgctgtgtgatgagcaggccccacagaggcagaggctgctggctgacctcctgcacaacgtcagccagaacatcgcggccgagagctatgtgagcgacatcaggcgcttcctcagtgcatttcacgagccacaggtggggctcatccaggccgcccagcagctgctgtgtgatgagcaggccccacagaggcagaggctgctggctgacctcctgcacaacgtcagccagaacatcgcggccgagacccgggctgaggacccgccgtggtttgaaggcttggagtcccgatttcagagcaagtctggctatctgagatacagctgtgagagccggatccggagttacctgagggaggtgagctcctacccctccacggtgggtgcggaggctcaggaggaattcctgcgggtcctcggctccatgtgccagaggctccggtccatgcagtacaatggcagctacttcgacagaggagccaagggcggcagccgcctctgcacaccggaaggctggttctcctgccagggtccctttgacatggacagctgcttatcaagacactccatcaacccctacagtaacagggagagcaggatcctcttcagcacctggaacctggatcacataatagaaaagaaacgcaccatcattcctacactggtggaagcaattaaggaacaagatggaagagaagtggactgggagtatttttatggcctgctttttacctcagagaacctaaaactagtgcacattgtctgccataagaaaaccacccacaagctcaactgtgacccaagcagaatctacaaaccccagacaaggttgaagcggaagcagcctgtgcggaaacgccag

SEQ ID NO: 30 - protein, 817 aa, synthetic construct
MEVTGDAGVPESGEIRTLKPCLLRRNYSREQHGVAASCLEDLRSKACDILAIDKSLTPVTLVLAEDGTIVDDDDYFLCLPSNTKFVALASNEKWAYNNSDGGTAWISQESFDVDETDSGAGLKWKNVARQLKEDLSSIILLSEEDLQMLVDAPCSDLAQELRQSCATVQRLQHTLQQVLDQREEVRQSKQLLQLYLQALEKEGSLLSKQEESKAAFGEEVDAVDTGISRETSSDVALASHILTALREKQAPELSLSSQDLELVTKEDPKALAVALNWDIKKTETVQEACERELALRLQQTQSLHSLRSISASKASPPGDLQNPKRARQDPTGSGSGSGSGGFNVLMVHKRSHTGERPLQCEICGFTCRQKGNLLRHIKLHTGEKPFKCHLCNYACQRRDALSGGGGEGRGSLLTCGDVEENPGPRMLQKPKSVKLWALRSPRKFGVAGRSCQEVLRKGCLRFQLPERGSRLCLYEDGTELTEDYFPSVPDNAALVLLTLGQAWQGYVSDIRRFLSAFHEPQVGLIQAAQQLLCDEQAPQRQRLLADLLHNVSQNIAAESYVSDIRRFLSAFHEPQVGLIQAAQQLLCDEQAPQRQRLLADLLHNVSQNIAAETRAEDPPWFEGLESRFQSKSGYLRYSCESRIRSYLREVSSYPSTVGAEAQEEFLRVLGSMCQRLRSMQYNGSYFDRGAKGGSRLCTPEGWFSCQGPFDMDSCLSRHSINPYSNRESRILFSTWNLDHIIEKKRTIIPTLVEAIKEQDGREVDWEYFYGLLFTSENLKLVHIVCHKKTTHKLNCDPSRIYKPQTRLKRKQPVRKRQ

SEQ ID NO: 31 - DNA, 180 bp, synthetic construct
  source 1..180: mol_type = other DNA
ttcaatgtcttaatggttcataagcgaagccatactggtgaacgcccattgcagtgcgaaatatgcggctttacctgccgccagaaaggtaacctcctccgccacattaaactgcacacaggggaaaaaccttttaagtgtcacctctgcaactatgcatgccaaagaagagatgcgctc

SEQ ID NO: 32 - protein, 60 aa, synthetic construct; identical to SEQ ID NO: 3

SEQ ID NO: 33 - protein, 60 aa, synthetic construct; identical to SEQ ID NO: 4

SEQ ID NO: 34 - DNA, 1179 bp, synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 35 - DNA, 503 bp, synthetic construct
  source 1..503: mol_type = other DNA
ttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgactcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaggg

SEQ ID NO: 36 - DNA, 180 bp, synthetic construct
  source 1..180: mol_type = other DNA
ttcaatgtcttaatggttcatcggcgaagccatactggtgaacgcccattgcagtgcgaaatatgcggctttacctgccgccagcgcggtaacctcctccgccacattcgtctgcacacaggggaacggccttttcggtgtcacctctgcaactatgcatgccaaagaagagatgcgctc